(19) United States
(12) Patent Application Publication
(10) Pub. No.: US 2017/0165286 A1
(43) Pub. Date: Jun. 15, 2017

(54) COMPOSITIONS AND METHODS FOR TREATING DIABETES AND LIVER DISEASES

(71) Applicant: Cempra Pharmaceuticals, Inc., Chapel Hill, NC (US)

(72) Inventor: Prabhavathi FERNANDES, Chapel Hill, NC (US)

(21) Appl. No.: 15/118,124

(22) PCT Filed: Feb. 11, 2015
(86) PCT No.: PCT/US15/15353

§ 371 Date: Aug. 11, 2016

Related U.S. Application Data
(60) Provisional application No. 61/939,961, filed on Feb. 14, 2014.
(60) Provisional application No. 61/984,225, filed on Apr. 25, 2014.
(60) Provisional application No. 62/086,911, filed on Dec. 3, 2014.

(51) Int. Cl. A61K 31/7056

(52) U.S. Cl. CPC A61K 31/7056

(57) ABSTRACT

Compounds, compositions, and methods are described herein for treating diabetes, fatty liver diseases, fibrotic diseases, such as liver and pulmonary fibrosis, and hepatocellular carcinoma.

### CROSS-REFERENCE TO RELATED APPLICATIONS

This application claims the benefit under 35 U.S.C. §119(e) of U.S. Provisional Application No. 61/939,961, filed Feb. 14, 2014, U.S. Provisional Application No. 61/984,225, filed Apr. 25, 2014, and U.S. Provisional Application No. 62/086,911, filed Dec. 3, 2014, the disclosures of each of which are expressly incorporated herein by reference.

### TECHNICAL FIELD

The invention described herein pertains to compounds, compositions, and methods for treating diabetes, fatty liver diseases, fibrotic diseases, such as liver and pulmonary fibrosis, and hepatocellular carcinoma.

### BACKGROUND AND SUMMARY OF THE INVENTION

Diabetes mellitus (DM), commonly referred to as diabetes, is a group of metabolic diseases characterized by prolonged periods of high blood sugar levels. If left untreated, diabetes can cause many complications, including acute complications such as diabetic ketoacidosis and nonketotic hyperosmolar coma, and serious long-term complications such as cardiovascular disease, stroke, kidney failure, foot ulcers and damage to the eyes. Diabetes is generally caused by either the pancreas not producing enough insulin or the cells of the body not responding properly to the insulin produced.

There are three main types of diabetes mellitus. Type 1 DM, also referred to as insulin-dependent diabetes mellitus (IDDM) or juvenile diabetes, results from the insufficient insulin production. Type 2 DM, also referred to as non insulin-dependent diabetes mellitus (NIDDM) or adult-onset diabetes, generally begins with insulin resistance, where cells fail to properly respond to insulin. Type 2 DM may lead to type 1 DM. Gestational diabetes, the third type, occurs when pregnant women without a previous history of diabetes develop high blood glucose levels.

An estimated 387 million people have diabetes worldwide, with type 2 diabetes accounting for about 90% of the cases. Diabetes is estimated to result in 2 to 5 million deaths per year. In addition, the number of people with diabetes is reportedly expected to continually rise year-after-year. The global economic cost of diabetes is estimated to be more than $600 billion, with United States diabetes costs topping $200 billion.

Insulin is the principal hormone that regulates the uptake of glucose from the blood into most cells of the body, especially liver, muscle, and adipose tissue. Insulin deficiency and/or insulin receptor insensitivity plays a central role in all forms of diabetes mellitus. The body obtains glucose from the intestinal absorption of food, the breakdown of glycogen stored in the liver, and gluconeogenesis, the generation of glucose from non-carbohydrate sources in the body. Insulin balances glucose levels in the body by inhibiting gluconeogenesis and/or the breakdown of glycogen. Insulin also stimulates glucose transport into fat and muscle cells, and stimulates the storage of glucose in the form of glycogen in the liver.

In response to rising levels of blood glucose, typically after eating, insulin is released into the blood by beta cells found in the islets of Langerhans in the pancreas. Lower glucose levels result in decreased insulin release from the beta cells and in the glucagon-mediated breakdown of glycogen to glucose. Thus, if insufficient insulin is available, cells respond poorly to the effects of insulin due to insulin insensitivity or insulin resistance, or the insulin itself is defective, then glucose will not be properly absorbed or appropriately stored in the liver and muscles. The net effect is persistently high levels of blood glucose, poor protein synthesis, acidosis, and other metabolic dysfunction, including glycosuria, polyuria and increased fluid loss, lost blood volume, dehydration and polydipsia.

In many cases, diabetes is comorbid with fatty liver disease (FLD), including for example, non-alcoholic steatohepatitis (NASH). However, the subject populations are not coextensive.

FLD, also referred to as fatty liver, is a reversible condition where large vacuoles of triglyceride fat accumulate in liver cells via the process of steatosis, an abnormal retention of lipids within a cell. Though FLD has multiple causes, two primary causes include excessive alcohol intake and obesity, with or without co-morbid insulin resistance. FLD also reportedly occurs with other diseases with fat metabolism dysfunction. Morphologically, regardless of the cause, including alcoholic FLD from nonalcoholic FLD, FLD generally shows microvesicular and macrovesicular fatty changes at different stages.

Though FLD itself may be reversible, the accumulation of fat may also be accompanied by a progressive hepatitis, inflammation of the liver, generally referred to as steatohepatitis, and lead to more severe nonalcoholic fatty liver disease (NAFLD), and the more severe NASH. In some instances where excessive alcohol intake is a contributor, FLD may also be termed alcoholic steatosis, or the more severe form alcoholic steatohepatitis (ASH).

NASH is a progressive form, and generally severe form, of NAFLD where accumulation of excessive fat (steatosis) coexists with liver cell injury, inflammation and fibrosis, which eventually leads to cirrhosis and hepatocellular carcinoma.

Generally, the pathology of FLD is the intracytoplasmatic accumulation of triglycerides (neutral fats). In early stages of the disease, the hepatocytes present small fat vacuoles (liposomes) around the nucleus (microvesicular fatty change). Liver cells are filled with multiple fat droplets that do not displace the centrally located nucleus. In later stages, the size of the vacuoles increases, pushing the nucleus to the periphery of the cell, giving characteristic signet ring appearance (macrovesicular fatty change). These vesicles are well delineated and optically “empty” because fats dissolve during tissue processing. Large vacuoles may coalesce and produce fatty cysts, and other irreversible lesions. Macrovesicular steatosis is reportedly the most common form of FLD and is typically associated with alcohol, diabetes, obesity and corticosteroids. Acute fatty liver of pregnancy and Reye's syndrome are examples of severe liver disease caused by microvesicular fatty change. Generally, the diagnosis of steatosis is made when fat in the liver exceeds 5-10% by weight.

FLD may be the result of one or multiple underlying causes, including alcohol and metabolic syndrome, diabetes, hypertension, obesity and dyslipidemia, metabolic causes, such as abetalipoproteinemia, glycogen storage diseases, Weber-Christian disease, acute fatty liver of pregnancy, and lipodystrophy, nutritional causes, such as, malnutrition, total parenteral nutrition, severe weight loss, refeeding syndrome, jejunoileal bypass, gastric bypass, and jejunal diverticulosis with bacterial overgrowth, drugs and toxin causes, such as may occur upon exposure or treatment with amiodarone, methotrexate, diltiazem, expired tetracycline, highly active antiretroviral therapy, glucocorticoids, tamoxifen, and environmental hepatotoxins like phosphorus or mushroom poisoning, and other causes, such as inflammatory bowel disease, HIV, hepatitis C, including genotype 3, and alpha 1-antitrypsin deficiency, and combinations thereof.

Defects in fatty acid metabolism may also be responsible for the pathogenesis of FLD, which may be due to imbalance in energy consumption and its combustion, resulting in lipid storage, or can be a consequence of peripheral resistance to insulin, whereby the transport of fatty acids from adipose tissue to the liver is increased. Impairment or inhibition of receptor molecules (PPAR-α, PPAR-γ and SREBP1) that control the enzymes responsible for the oxidation and synthesis of fatty acids has also been reported to contribute to fat accumulation. In addition, alcoholism is reported to damage mitochondria and other cellular structures, further impairing cellular energy mechanism. Nonalcoholic FLD may arise from an excess of unmetabolised energy in liver cells. Hepatic steatosis is reportedly reversible and to some extent nonprogressive if the underlying cause is reduced or removed.

In many cases, NASH is comorbid with diabetes, but the subject population is not coextensive.

Progression of steatohepatitis to alcoholic steatohepatitis (ASH) or non-alcoholic steatohepatitis (NASH) reportedly depends on the persistence or severity of the inciting cause. Pathological lesions in both conditions are similar. However, the extent of inflammatory response varies widely and does not always correlate with the degree of fat accumulation. Steatosis (retention of lipid) and onset of steatohepatitis may represent successive stages in FLD progression.

Liver disease with extensive inflammation and a high degree of steatosis often progresses to more severe forms of the disease. Hepatocyte ballooning and necrosis of varying degrees are often present at this stage. Liver cell death and inflammatory responses lead to the activation of stellate cells, which play an important role in hepatic fibrosis. The extent of fibrosis varies widely. Perisinusoidal fibrosis is reportedly most common, especially in adults, and predominates in zone 3 around the terminal hepatic veins.

The further progression to cirrhosis may be influenced by the amount of fat and degree of steatohepatitis and by a variety of other sensitizing factors. In alcoholic FLD, the transition to cirrhosis related to continued alcohol consumption is well documented, but the process involved in nonalcoholic FLD is less clear.

Finally, left untreated, fatty liver disease, including NASH, often progresses to hepatocellular carcinoma (HCC). It has been reported that non-alcoholic fatty liver disease (NAFLD) is highly prevalent (15% to 45%) in modern societies. In addition, it has been reported that 10% to 25% of those cases develop hepatic fibrosis leading to cirrhosis, end-stage liver disease or HCC. To date, no single therapy has been approved for treating NAFLD or NASH. Accordingly, there exists a need for new compounds, pharmaceutical compositions thereof, and methods for using the same to treat patients with fatty liver disease.

Pulmonary fibrosis, or scarring of the lung, is the formation or development of excess fibrous connective tissue (fibrosis) in the lungs. Pulmonary fibrosis involves gradual exchange of normal lung parenchyma with fibrotic tissue. The replacement of normal lung with scar tissue causes irreversible decrease in oxygen diffusion capacity. In addition, decreased compliance makes pulmonary fibrosis a restrictive lung disease. It is the main cause of restrictive lung disease that is intrinsic to the lung parenchyma. Five million people worldwide are affected by pulmonary fibrosis. A wide range of incidence and prevalence rates have been reported for pulmonary fibrosis.

Pulmonary fibrosis may be a secondary effect of other diseases. Most of these are classified as interstitial lung diseases. Examples include autoimmune disorders, viral infections or other microscopic injuries to the lung. However, pulmonary fibrosis can also be idiopathic, and appear without any known cause. Most idiopathic cases are diagnosed as idiopathic pulmonary fibrosis. This is a diagnosis of exclusion of a characteristic set of histologic/pathologic features known as usual interstitial pneumonia (UIP). In either case, there is a growing body of evidence which points to a genetic predisposition in a subset of patients. For example, a mutation in surfactant protein C (SP-C) has been found to exist in some families with a history of pulmonary fibrosis.

Diseases and conditions that may cause pulmonary fibrosis as a secondary effect include inhalation of environmental and occupational pollutants, such as in asbestosis, silicosis and exposure to certain gases; hypersensitivity pneumonitis, most often resulting from inhaling dust contaminated with bacterial, fungal, or animal products; cigarette smoking; connective tissue diseases, such as rheumatoid arthritis, SLE, and scleroderma; diseases that involve connective tissue, such as sarcoidosis and Wegener's granulomatosis; and infections.

Pulmonary fibrosis as a secondary effect may also be caused by certain medications, such as amiodarone, bleomycin (pingyangmycin), busulfan, methotrexate, and nitrofurantoin. Pulmonary fibrosis as a secondary effect may also be caused by radiation therapy to the chest.

Pulmonary fibrosis creates scar tissue. The scarring is permanent once it has developed. Therefore, treatment is generally limited to slowing the progression and prevention by removing or limiting underlying causes. Treatment options for idiopathic pulmonary fibrosis are very limited. Though research trials are ongoing, there is no evidence that any medications can significantly help this condition. Lung transplantation is the only therapeutic option available in severe cases. Since some types of lung fibrosis can respond to corticosteroids (such as prednisone) and/or other medications that suppress the body's immune system, these types of drugs are sometimes prescribed in an attempt to slow the processes that lead to fibrosis. Nonetheless, currently there are no known treatments or cure options for idiopathic pulmonary fibrosis. Accordingly, new treatment options are needed.

Described herein are compounds, pharmaceutical compositions thereof, and methods and uses of the foregoing for treating host animals with diabetes. Also described herein are compounds, pharmaceutical compositions thereof, and methods and uses of the foregoing for treating host animals with FLDs. Also described herein are compounds, pharmaceutical compositions thereof, and methods and uses of the foregoing for treating host animals with pulmonary fibrosis and/or liver fibrosis, including the prophylactic treatment of pulmonary fibrosis and/or liver fibrosis. Also described herein are compounds, pharmaceutical compositions thereof, and methods and uses of the foregoing for the prophylactic treatment of host animals at risk of developing HCC.

Also described herein are pharmaceutical compositions, including unit doses and unit dosage forms, containing one or more of the compounds described herein. In one aspect, the compositions include a therapeutically effective amount of the one or more compounds for treating a host animal with diabetes, FLDs, fibrotic diseases, and/or HCC, including prophylactically treating pulmonary or liver fibrosis, and/or prophylactically treating HCC. It is to be understood that the compositions may include other components and/or ingredients, including, but not limited to, other therapeutically active compounds, and/or one or more carriers, diluents, excipients, and the like, and combinations thereof. In another embodiment, methods for using the compounds and pharmaceutical compositions for treating host animals with diabetes, FLDs, fibrotic diseases, and/or HCC, including prophylactically treating pulmonary or liver fibrosis, and/or prophylactically treating HCC are also described herein. In one aspect, the methods include the step of administering one or more of the compounds and/or compositions described herein to a host animal with diabetes, FLDs, fibrotic diseases, and/or HCC, including prophylactically treating pulmonary or liver fibrosis, and/or prophylactically treating HCC. In another aspect, the methods include administering a therapeutically effective amount of the one or more compounds and/or compositions described herein for treating host animals with diabetes, FLDs, fibrotic diseases, and/or HCC, including prophylactically treating pulmonary or liver fibrosis, and/or prophylactically treating HCC. In another embodiment, uses of the compounds and compositions described herein in the manufacture of a medicament for treating host animals with diabetes, FLDs, fibrotic diseases, and/or HCC, including prophylactically treating pulmonary or liver fibrosis, and/or prophylactically treating HCC are also described herein. In one aspect, the medicaments include a therapeutically effective amount of the one or more compounds and/or compositions described herein for treating a host animal with diabetes, FLDs, fibrotic diseases, and/or HCC, including prophylactically treating pulmonary or liver fibrosis, and/or prophylactically treating HCC.

It is to be understood that the compounds, compositions, and methods and uses for treating diabetes, FLDs, fibrotic diseases, and/or HCC may be used in conjunction with other treatments, including treating the underlying cause, such as decreasing the excess consumption of alcohol, and/or prolonged diet containing foods with a high proportion of calories coming from lipids and/or carbohydrates, administering medications that decrease insulin resistance, hyperlipidemia, and/or induce weight loss to improve liver function. It is also to be understood that the compounds, compositions, and methods and uses for treating pulmonary fibrosis may be used in conjunction with other treatments, including treating the underlying cause, such as decreasing exposure to substances and materials that may result in pulmonary fibrosis, or removing the conditions or scenarios that may result in pulmonary fibrosis. It is also to be understood herein that the compounds described herein may be used alone or in combination with other compounds useful for treating such diseases, including those compounds that may be therapeutically effective by the same or different modes of action. In addition, it is to be understood herein that the compounds described herein may be used in combination with other compounds that are administered to treat other symptoms of such disease, such as compounds administered to treat obesity, and the like.

### BRIEF DESCRIPTION OF THE DRAWINGS

FIG. 1 shows liver weight in normal, vehicle treated, and solithromycin treated test animals.

FIG. 2 shows whole blood glucose levels in normal, vehicle treated, and solithromycin treated test animals.

FIG. 3 shows serum chylomicron levels in normal, vehicle treated, and solithromycin treated test animals.

FIG. 4 shows serum VLDL-cholesterol levels in normal, vehicle treated, and solithromycin treated test animals.

FIG. 5 shows serum HDL-cholesterol levels in normal, vehicle treated, and solithromycin treated test animals.

FIG. 6 shows serum triglyceride levels in normal, vehicle treated, and solithromycin treated test animals.

FIG. 7 shows plasma MIF levels in normal, vehicle treated, and solithromycin treated test animals.

FIG. 8 shows the NAFLD Activity Score (NAS) in normal, vehicle treated, and solithromycin treated test animals.

FIG. 9 shows the Sirius red-positive area in normal, vehicle treated, and solithromycin treated test animals.

FIG. 10 shows G6pc mRNA expression levels in normal, vehicle treated, and solithromycin treated test animals.

FIG. 11 shows FBPase mRNA expression levels in normal, vehicle treated, and solithromycin treated test animals.

### DETAILED DESCRIPTION

It has been unexpectedly discovered that compounds, compositions, and methods described herein are useful for treating diabetes, FLDs, fibrotic diseases, and/or HCC, including prophylactically treating pulmonary or liver fibrosis, and/or prophylactically treating HCC.

Compounds, compositions, methods, and uses described herein unexpectedly decrease whole blood glucose levels in host animals. Compounds, compositions, methods, and uses described herein unexpectedly decrease liver weight and/or decrease liver-to-body weight ratio in host animals with FLD. Compounds, compositions, methods, and uses described herein unexpectedly suppress fat accumulation in the liver, which may lead to pale-yellow coloration of the liver in host animals with FLD. Compounds, compositions, methods, and uses described herein unexpectedly decrease, prevent, or slow the onset of liver fibrosis in host animals with FLD. Compounds, compositions, methods, and uses described herein unexpectedly decrease, prevent, or slow the onset of liver cancer such as HCC in host animals with FLD. Compounds, compositions, methods, and uses described herein unexpectedly decrease, prevent, or slow the onset of lung fibrosis in host animals.

In one illustrative embodiment of the invention, compounds, compositions thereof, and treatment methods using the foregoing are described herein for diabetes. In another embodiment, compounds, compositions thereof, and treatment methods using the foregoing are described herein for treating FLD. In another embodiment, the FLD is selected from NAFLD, ASH, and NASH, or a combination thereof. In another embodiment, the FLD is NASH. In another embodiment, compounds, compositions thereof, and treatment methods using the foregoing are described herein for treating diabetes. In another embodiment, the diabetes is type 1 DM. In another embodiment, the diabetes is type 2 DM. In another embodiment, the diabetes is gestational DM. In another embodiment, compounds, compositions thereof, and treatment methods using the foregoing are described herein for treating fibrotic diseases. In another embodiment, compounds, compositions thereof, and treatment methods using the foregoing are described herein for prophylactically treating HCC. In another embodiment, compounds, compositions thereof, and treatment methods using the foregoing are described herein for preventing and/or delaying the onset of HCC.

According to the methods described herein, the compounds may be administered directly or as part of a pharmaceutical composition, unit dose, or unit dosage form that may include one or more carriers, diluents, or excipients, or combinations thereof. In addition, the compounds, compositions, unit doses, and unit dosage forms described herein are useful for treating the foregoing diseases, and are used in the manufacture of medicaments for treating the foregoing diseases.

It has also been observed that compounds described herein accumulate in liver tissue. Compounds described herein unexpectedly do not cause liver damage, and are even well-tolerated by host animals, including humans, with mild, moderate, or even severe hepatic insufficiency. Accordingly, because compounds described herein can target the liver, they are useful for treating liver diseases, including all types of FLDs. It is therefore to be understood that the invention described herein does not include compounds that exhibit high hepatotoxicity at therapeutic doses, such as may occur with high doses of erythromycin or clarithromycin. Without being bound by theory, it is believed herein that the efficacy of the compounds described herein is not due to antibacterial effects, such as antibacterial effects on intestinal microflora. For example, compounds described herein do not affect Gram-negative enterics, such as Enterobacteriaceae, Gram-negative anaerobes, or endotoxin producing bacteria, and have a minimal effect on bowel flora.

It has also been unexpectedly discovered that compounds described herein, and compositions thereof, accumulate in the lung, and achieve high concentrations in the lung, in the epithelial lining fluid, as well as in pulmonary macrophages in human studies. In another embodiment, compounds, compositions thereof, and treatment methods using the foregoing are described herein for treating pulmonary fibrosis. In another embodiment, compounds, compositions thereof, and treatment methods using the foregoing are described herein for prophylactic treatment, such as for preventing and/or delaying the onset of pulmonary fibrosis. It is appreciated that under certain circumstances, there will be advanced notice that the host animal will be exposed to conditions or materials that may result in pulmonary fibrosis. Under those circumstances, compounds, compositions, and methods described herein may be used to prophylactically treat the host animal to either decrease the degree to which pulmonary fibrosis may occur, or alternatively, prevent the occurrence of pulmonary fibrosis following such exposure to pulmonary fibrosis causing conditions or materials. Without being bound by theory, it is believed herein that such prophylactic treatment may decrease the amount or extent of inflammation that may occur following such exposure to pulmonary fibrosis causing conditions or materials.

In another embodiment, compounds, compositions thereof, and treatment methods using the foregoing are described herein for prophylactically treating pulmonary fibrosis in immunosuppressed host animals, including humans. For example, patients undergoing or scheduled for lung transplantation may be administered immunosuppressants to counter rejection of the transplanted tissue. Such rejection has been reported to be preceded by or cause inflammation and/or fibrosis. Those patients may be administered compounds and compositions described herein before, or in conjunction with the transplantation procedure to prevent fibrosis.

In another embodiment, compounds, compositions, and methods are described herein for treating bronchiolitis, including panbronchiolitis, diffuse panbronchiolitis (DPB), bronchiectasis, and the like, in a host animal.

Several illustrative embodiments of the invention are described by the following clauses:

A composition, unit dose, or unit dosage form comprising one or more compounds of the formula

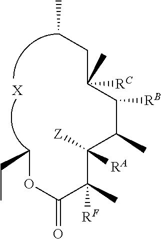

or a pharmaceutically acceptable salt thereof, or a hydroxyl or amino prodrug thereof; wherein:

X is a divalent radical selected from the group consisting of

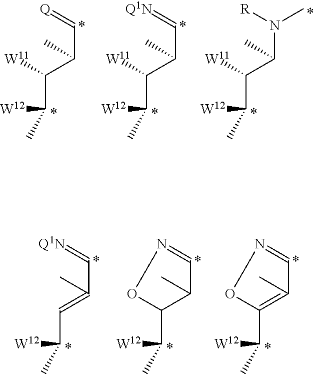

where X is connected at each (*) atom;

W¹¹ is hydroxy or a derivative thereof; W¹² is H, or hydroxy or a derivative thereof; or W¹¹ and W¹² are taken together with the attached carbon atoms to form an oxygen and/or nitrogen containing heterocycle, each of which is optionally substituted;

Q is O or (NR, H); where R is hydrogen or optionally substituted alkyl; or R and W11 are taken together to form an aminal ether, such as an optionally substituted 1,3-oxazine; and Q¹ is hydroxy or a derivative thereof or amino or a derivative thereof;

R^{A} is hydroxy or a hydroxy derivative, or a saccharide attached at oxygen; and Z is hydrogen; or R^{A} and Z are taken together with the attached carbon to form a C═O group.

R^{B} is an amino containing saccharide;

R^{C} is hydroxy or a derivative thereof; or

R^{C} and Q are taken together to form an enol ether; or

R^{C} and W¹² and Q are taken together to form a ketal; and

R^{F} is H or F.

The composition, unit dose, or unit dosage form of the preceding clause wherein at least one compound is of the formula

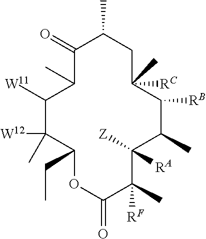

or a pharmaceutically acceptable salt thereof.

The composition, unit dose, or unit dosage form of the preceding clause wherein at least one compound is of the formula

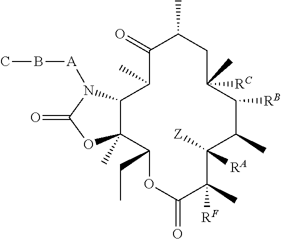

or a pharmaceutically acceptable salt thereof; where where C is H or alkyl, alkenyl, alkynyl, heteroalkyl, aryl, heteroaryl, arylalkyl, or heteroarylalkyl, each of which is optionally substituted;

B is a bond, or B is an optionally substituted heteroaryl; and

A is a bond, or A is an optional linker formed from O, C(O), CR, CR₂, and NR, and combinations thereof, where each R is independently selected in each instance from being absent to form a double or triple bond, being hydrogen, or being an optionally substituted alkyl.

The composition, unit dose, or unit dosage form of any one of the preceding clauses wherein at least one compound is of the formula

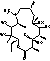

or a pharmaceutically acceptable salt thereof.

A method for treating diabetes in a host animal, the method comprising the step of administering to the host animal an effective amount of the composition, unit dose, or unit dosage form of any one of the preceding clauses.

A method for treating a FLD in a host animal, the method comprising the step of administering to the host animal an effective amount of the composition, unit dose, or unit dosage form of any one of the preceding clauses.

A method for treating NASH in a host animal, the method comprising the step of administering to the host animal an effective amount of the composition, unit dose, or unit dosage form of any one of the preceding clauses.

A method for treating liver fibrosis in a host animal, the method comprising the step of administering to the host animal an effective amount of the composition, unit dose, or unit dosage form of any one of the preceding clauses.

A method for prophylactically treating liver fibrosis in a host animal, the method comprising the step of administering to the host animal an effective amount of the composition, unit dose, or unit dosage form of any one of the preceding clauses.

A method for prophylactically treating HCC in a host animal, the method comprising the step of administering to the host animal an effective amount of the composition, unit dose, or unit dosage form of any one of the preceding clauses.

A method for treating lung fibrosis in a host animal, the method comprising the step of administering to the host animal an effective amount of the composition, unit dose, or unit dosage form of any one of the preceding clauses.

A method for prophylactically treating lung fibrosis in a host animal, the method comprising the step of administering to the host animal an effective amount of the composition, unit dose, or unit dosage form of any one of the preceding clauses.

Use of the composition, unit dose, or unit dosage form of any one of the preceding clauses in the manufacture of a medicament for treating diabetes in a host animal.

Use of the composition, unit dose, or unit dosage form of any one of the preceding clauses in the manufacture of a medicament for treating a FLD in a host animal.

Use of the composition, unit dose, or unit dosage form of any one of the preceding clauses in the manufacture of a medicament for treating NASH in a host animal.

Use of the composition, unit dose, or unit dosage form of any one of the preceding clauses in the manufacture of a medicament for treating or prophylactically treating liver fibrosis in a host animal.

Use of the composition, unit dose, or unit dosage form of any one of the preceding clauses in the manufacture of a medicament for prophylactically treating HCC in a host animal.

Use of the composition, unit dose, or unit dosage form of any one of the preceding clauses in the manufacture of a medicament for treating or prophylactically treating lung fibrosis in a host animal.

The unit dose, unit dosage form, method, use, or composition of any one of the preceding clauses wherein Z is H and R^{A} is hydroxy or a derivative thereof.

The unit dose, unit dosage form, method, use, or composition of any one of the preceding clauses wherein Z is H and R^{A} is hydroxy or acyloxy.

The unit dose, unit dosage form, method, use, or composition of any one of the preceding clauses wherein Z is H and R^{A} is cladinosyl.

The unit dose, unit dosage form, method, use, or composition of any one of the preceding clauses wherein Z is H and R^{A} is not cladinosyl.

The unit dose, unit dosage form, method, use, or composition of any one of the preceding clauses wherein Z and R^{A} are taken together with the attached carbon to form C═O.

The unit dose, unit dosage form, method, use, or composition of any one of the preceding clauses wherein R^{B} is desosaminyl.

The unit dose, unit dosage form, method, use, or composition of any one of the preceding clauses wherein R^{B} is not desosaminyl.

The unit dose, unit dosage form, method, use, or composition of any one of the preceding clauses wherein R^{B} is a desosaminyl derivative.

The unit dose, unit dosage form, method, use, or composition of any one of the preceding clauses wherein the desosaminyl derivative has modified hydrogen bonding compared to desosaminyl.

The unit dose, unit dosage form, method, use, or composition of any one of the preceding clauses wherein the desosaminyl derivative has modified basicity compared to desosaminyl.

The unit dose, unit dosage form, method, use, or composition of any one of the preceding clauses wherein the nitrogen of the desosaminyl derivative has modified hydrogen bonding compared to desosaminyl.

The unit dose, unit dosage form, method, use, or composition of any one of the preceding clauses wherein the nitrogen of the desosaminyl derivative has modified basicity compared to desosaminyl.

The unit dose, unit dosage form, method, use, or composition of any one of the preceding clauses wherein the oxygen of the desosaminyl derivative has modified hydrogen bonding compared to desosaminyl.

The unit dose, unit dosage form, method, use, or composition of any one of the preceding clauses wherein R^{B} is O-acyl desosaminyl.

The unit dose, unit dosage form, method, use, or composition of any one of the preceding clauses wherein R^{B} is O-alkyl desosaminyl.

The unit dose, unit dosage form, method, use, or composition of the preceding clause wherein the alkyl is C₂-C₁₈ alkyl.

The unit dose, unit dosage form, method, use, or composition of any one of the preceding clauses wherein R^{B} is desosaminyl-N-oxide or desmethyl desosaminyl-N-oxide.

The unit dose, unit dosage form, method, use, or composition of any one of the preceding clauses wherein R^{B} is desmethyl desosaminyl.

The unit dose, unit dosage form, method, use, or composition of any one of the preceding clauses wherein R^{B} is N-acyl desmethyl desosaminyl.

The unit dose, unit dosage form, method, use, or composition of any one of the preceding clauses wherein R^{B} is N-substituted desmethyldesosaminyl, where the substituent is ethyl, hydroxyethyl, propyl, isopropyl, hydroxypropyl, butyl, isobutyl, sec-butyl, hydroxy-sec-butyl, cyclobutyl, cyclopropylmethyl, cyclopentylmethyl, or propargyl.

The unit dose, unit dosage form, method, use, or composition of any one of the preceding clauses wherein R^{B} is N-substituted bisdesmethyldesosaminyl, where the substituent is ethyl, hydroxyethyl, propyl, isopropyl, hydroxypropyl, butyl, isobutyl, sec-butyl, hydroxy-sec-butyl, cyclobutyl, cyclopropylmethyl, cyclopentylmethyl, or propargyl.

The unit dose, unit dosage form, method, use, or composition of any one of the preceding clauses wherein the substituent is isopropyl, isobutyl, cyclopropylmethyl, cyclopentylmethyl, or cyclobutyl.

The unit dose, unit dosage form, method, use, or composition of any one of the preceding clauses wherein the substituent is isopropyl, cyclobutyl, 2-hydroxypropyl, or 2-hydroxy-2-methylpropyl.

The unit dose, unit dosage form, method, use, or composition of any one of the preceding clauses wherein R^{B} is N-alkyl desmethyl desosaminyl, where the alkyl is C₂-C₁₈ alkyl.

The unit dose, unit dosage form, method, use, or composition of any one of the preceding clauses wherein R^{B} is desmethyl desosaminyl.

The unit dose, unit dosage form, method, use, or composition of any one of the preceding clauses wherein R^{B} is N-ethyl desmethyl desosaminyl.

The unit dose, unit dosage form, method, use, or composition of any one of the preceding clauses wherein R^{B} is N-(2-hydroxyethyl) desmethyl desosaminyl.

The unit dose, unit dosage form, method, use, or composition of any one of the preceding clauses wherein R^{B} is N-propargyl desmethyl desosaminyl.

The unit dose, unit dosage form, method, use, or composition of any one of the preceding clauses wherein R^{B} is a radical of the formula

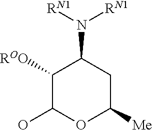

where each R^{N1} is independently selected in each instance from H and acyl, and alkyl, cycloalkyl, arylalkyl, and heteroarylalkyl, each of which is optionally substituted, providing that at least one R^{N1} is not methyl; or both R^{N1} are taken together with the attached nitrogen to form a nitrogen containing heterocycle; and R^{O} is H or acyl, or alkyl, cycloalkyl, arylalkyl, and heteroarylalkyl, each of which is optionally substituted; or R^{O} and one R^{N1} are taken together with the attached atoms to form an oxygen and nitrogen containing heterocycle.

The unit dose, unit dosage form, method, use, or composition of any one of the preceding clauses wherein the heterocycle is a 5 to 7 member ring heterocycle.

The unit dose, unit dosage form, method, use, or composition of any one of the preceding clauses wherein R^{N1} and R^{O} are taken together to form a oxazolidinone, or an imino oxazolidinone of the formula

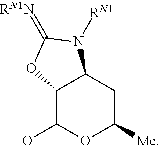

The unit dose, unit dosage form, method, use, or composition of any one of the preceding clauses wherein R^{C} is hydroxy or alkoxy.

The unit dose, unit dosage form, method, use, or composition of any one of the preceding clauses wherein R^{C} is hydroxy.

The unit dose, unit dosage form, method, use, or composition of any one of the preceding clauses wherein R^{C} is methoxy.

The unit dose, unit dosage form, method, use, or composition of any one of the preceding clauses wherein R^{F} is F.

The unit dose, unit dosage form, method, use, or composition of any one of the preceding clauses wherein Q¹ is hydroxy or a derivative thereof or amino or a derivative thereof.

The unit dose, unit dosage form, method, use, or composition of any one of the preceding clauses wherein Q¹ is hydroxy or a derivative thereof.

The unit dose, unit dosage form, method, use, or composition of any one of the preceding clauses wherein Q¹ is hydroxy or acyloxy.

The unit dose, unit dosage form, method, use, or composition of any one of the preceding clauses wherein W¹¹ and W¹² are both hydroxy.

The unit dose, unit dosage form, method, use, or composition of any one of the preceding clauses wherein W¹¹ and W¹² are taken together to form a carbonate.

The unit dose, unit dosage form, method, use, or composition of any one of the preceding clauses wherein W¹¹ is OH; and W¹² is H.

The unit dose, unit dosage form, method, use, or composition of any one of the preceding clauses wherein W¹¹ and W¹² are taken together with the attached carbon atoms to form to carbamate where the nitrogen thereof is substituted with a radical of the formula C-B-A-, where C is H or alkyl, alkenyl, alkynyl, heteroalkyl, aryl, heteroaryl, arylalkyl, or heteroarylalkyl, each of which is optionally substituted; B is a bond, or B is an optionally substituted heteroaryl; and A is a bond, or A is an optional linker formed from O, C(O), CR, CR₂, and NR, and combinations thereof, where each R is independently selected in each instance from being absent to form a double or triple bond, being hydrogen, or being an optionally substituted alkyl.

The unit dose, unit dosage form, method, use, or composition of any one of the preceding clauses wherein A is alkylene, such as C₃-C₅ alkylene, or C₄ alkylene, or (CH₂)₄. The unit dose, unit dosage form, method, use, or composition of any one of the preceding clauses wherein B is an imidazole radical. The unit dose, unit dosage form, method, use, or composition of any one of the preceding clauses wherein B is a 1,2,3-triazole radical. The unit dose, unit dosage form, method, use, or composition of any one of the preceding clauses wherein C is an optionally substituted heteroaryl or optionally substituted heteroarylalkyl radical. The unit dose, unit dosage form, method, use, or composition of any one of the preceding clauses wherein C is an optionally substituted phenyl. The unit dose, unit dosage form, method, use, or composition of any one of the preceding clauses wherein C is an amino substituted phenyl, such as 3-amino substituted phenyl.

The unit dose, unit dosage form, method, use, or composition of any one of the preceding clauses wherein the compound is a 14-ring macrocycle.

The unit dose, unit dosage form, method, use, or composition of any one of the preceding clauses wherein the compound is a 15-ring macrocycle.

The unit dose, unit dosage form, method, use, or composition of any one of the preceding clauses wherein the compound is selected from the group consisting of solithromycin, roxithromycin, azithromycin, flurithromycin, and dirithromycin, and pharmaceutically acceptable salts thereof, and combinations thereof.

The unit dose, unit dosage form, method, use, or composition of any one of the preceding clauses wherein the compound is roxithromycin or a pharmaceutically acceptable salt thereof.

The unit dose, unit dosage form, method, use, or composition of any one of the preceding clauses wherein the compound is solithromycin or a pharmaceutically acceptable salt thereof.

The unit dose, unit dosage form, method, use, or composition of any one of the preceding clauses wherein the compound has an average MIC₅₀ of about 4 or greater against erythromycin susceptible bacteria.

The unit dose, unit dosage form, method, use, or composition of any one of the preceding clauses wherein the compound has an average MIC₅₀ of about 8 or greater against erythromycin susceptible bacteria.

The unit dose, unit dosage form, method, use, or composition of any one of the preceding clauses wherein the compound has an average MIC₅₀ of about 16 or greater against erythromycin susceptible bacteria.

The unit dose, unit dosage form, method, use, or composition of any one of the preceding clauses wherein the compound has an average MIC₅₀ of about 32 or greater against erythromycin susceptible bacteria.

The unit dose, unit dosage form, method, use, or composition of any one of the preceding clauses wherein the compound is substantially nonantibacterial or does not have clinically significant antibacterial activity or efficacy.

The unit dose, unit dosage form, method, use, or composition of any one of the preceding clauses wherein the compound exhibits low hepatotoxicity.

The unit dose, unit dosage form, method, use, or composition of any one of the preceding clauses wherein the compound does not exhibit negative QT effects, such as QT and/or tQT prolongation.

The unit dose, unit dosage form, method, use, or composition of any one of the preceding clauses wherein the compound does not increase alanine transaminase (ALT) to a clinically significant level.

The unit dose, unit dosage form, method, use, or composition of any one of the preceding clauses wherein the compound does not increase aspartate transaminase (AST) to a clinically significant level.

The unit dose, unit dosage form, method, use, or composition of any one of the preceding clauses wherein the compound decreases serum chylomicron.

The unit dose, unit dosage form, method, use, or composition of any one of the preceding clauses wherein the compound decreases serum VLDL-cholesterol.

The unit dose, unit dosage form, method, use, or composition of any one of the preceding clauses wherein the compound decreases serum LDL-cholesterol.

The unit dose, unit dosage form, method, use, or composition of any one of the preceding clauses wherein the compound increases serum HDL-cholesterol.

The unit dose, unit dosage form, method, use, or composition of any one of the preceding clauses wherein the method results in lower whole blood glucose levels.

The unit dose, unit dosage form, method, use, or composition of any one of the preceding clauses wherein the method results in decreasing hyperglycemia.

The unit dose, unit dosage form, method, use, or composition of any one of the preceding clauses wherein the method results in suppressing hepatic gluconeogenesis.

The unit dose, unit dosage form, method, use, or composition of any one of the preceding clauses wherein the method results in suppressing hepatic gluconeogenesis and/or decreasing inflammation.

The unit dose, unit dosage form, method, use, or composition of any one of the preceding clauses wherein the method results in decreasing expression (mRNA levels) or the level of glucose-6-phosphatase (G6pc).

The unit dose, unit dosage form, method, use, or composition of any one of the preceding clauses wherein the method results in decreasing expression (mRNA levels) or the level of fructose 1,6 biphosphatase (FBPase)

The unit dose, unit dosage form, method, use, or composition of any one of the preceding clauses wherein the method results in decreased fat deposition in the liver.

The unit dose, unit dosage form, method, use, or composition of any one of the preceding clauses wherein the method results in suppressed pale-yellow coloration of the liver.

The unit dose, unit dosage form, method, use, or composition of any one of the preceding clauses wherein the method results in decreased liver weight.

The unit dose, unit dosage form, method, use, or composition of any one of the preceding clauses wherein the method results in decreased liver-to-body weight ratio.

The unit dose, unit dosage form, method, use, or composition of any one of the preceding clauses wherein the method results in decreased ballooning, decreased hepatic ballooning degeneration, and/or a decreased ballooning score.

The unit dose, unit dosage form, method, use, or composition of any one of the preceding clauses wherein the method results in a ballooning decrease of about 50%, about 70%, about 80% about 90%, about 95%, or more. It is understood that ballooning may be expressed as a score from 0-10, and therefore, the foregoing percentage improvements are expressed in the corresponding score, such as a change in score of 1, 2, 3, 4, or 5, and the like. It is to be understood, however, that therapeutic efficacy is established when ballooning decreases by as little as a single index, such as from 10 to 9, from 5 to 4, or from 2 to 1, and the like.

The unit dose, unit dosage form, method, use, or composition of any one of the preceding clauses wherein the method results in substantially complete absence of ballooning.

The unit dose, unit dosage form, method, use, or composition of any one of the preceding clauses wherein the method results in decreased steatosis.

The unit dose, unit dosage form, method, use, or composition of any one of the preceding clauses wherein the method results in decreased liver fibrosis.

The unit dose, unit dosage form, method, use, or composition of any one of the preceding clauses wherein the method results in increase plasma MIF.

The unit dose, unit dosage form, method, use, or composition of any one of the preceding clauses wherein the method results in decreased lung fibrosis.

The unit dose, unit dosage form, method, use, or composition of any one of the preceding clauses wherein the method results in decreased neutrophil infiltration.

The unit dose, unit dosage form, method, use, or composition of any one of the preceding clauses wherein the host animal has hepatic insufficiency.

The unit dose, unit dosage form, method, use, or composition of any one of the preceding clauses wherein the treatment is prophylactic or therapeutic for a host animal that will undergo or is undergoing exposure to radiation, such as radiation therapy.

The unit dose, unit dosage form, method, use, or composition of any one of the preceding clauses wherein the treatment is prophylactic or therapeutic for a cancer patient that will undergo or is undergoing exposure to radiation, such as radiation therapy. Without being bound by theory, it is believed herein that substantial lung inflammation occurs with radiation therapy. Therefore, compounds, compositions, and methods described herein can be used both in advance of such treatment, and post treatment.

The unit dose, unit dosage form, method, use, or composition of any one of the preceding clauses wherein the treatment is prophylactic or therapeutic for a host animal that will undergo immunosuppression therapy and/or lung transplant.

The unit dose, unit dosage form, method, use, or composition of any one of the preceding clauses wherein the method does not result in negative effects on whole animal body weight.

The unit dose, unit dosage form, method, use, or composition of any one of the preceding clauses wherein the administering step includes a dose of about 0.1-10, 0.1-5, or 0.3-2 mg/kg bid

The unit dose, unit dosage form, method, use, or composition of any one of the preceding clauses wherein the administering step includes a daily dose of about 1-600, about 5-500, about 10-400, about 100-400, about 200-400, about 150-350, about 50-300, about 100-300, about 150-250, about 50-200, or about 100-200 mg administered q.d. or b.i.d.

The unit dose, unit dosage form, method, use, or composition of any one of the preceding clauses wherein the administering step includes a daily dose of about 500, about 450, about 400, about 350, about 300, about 250, about 200, about 150, or about 100 mg administered q.d. or b.i.d.

The unit dose, unit dosage form, method, use, or composition of any one of the preceding clauses wherein the administering step includes a daily dose of about 300, about 275, about 260, about 240, about 225, about 200, about 175, about 150, or about 125 mg administered q.d. or b.i.d.

The unit dose, unit dosage form, method, use, or composition of any one of the preceding clauses wherein the administering step is b.i.d.

The unit dose, unit dosage form, method, use, or composition of any one of the preceding clauses wherein the administering step is q.d.

It is appreciated and understood herein that patients suffering from NASH may suffer mortality arising from cardiovascular death arising as a consequence of NASH. Therefore, it is understood that compounds that also have the added feature of not causing unwanted cardiovascular effects, such as QT effects, are desirable.

In another embodiment, compounds and methods described herein do not have to be used in conjunction with lipid management therapy, such as co-administration of statins, and the like.

It has been discovered herein that NASH may be treated by decreasing under certain conditions inflammation of the liver. It has also been discovered herein that compounds described herein decrease liver inflammation. Accordingly, though without being bound by theory, it is believed herein that the efficacy of the compounds in treating NASH arise at least in part from an anti-inflammatory effect of the compounds that are administered according to the compositions and methods described herein.

It has also been discovered herein that compounds described herein accumulate in liver tissue. It is appreciated that in many therapy areas, liver clearance is the less desirable pathway due to the potential for adverse effects on the liver due to accumulation of the therapeutic agent. However, it has been discovered that at doses therapeutic for treating the diseases described herein, compounds described herein accumulate sufficiently in the liver to be effective, but not to levels so high to cause significant adverse effects.

It has been discovered that compounds described herein have potent anti-inflammatory activities. Without being bound by theory, it is believed herein that the efficacy of compounds, compositions, methods, and uses described herein is due at least in part to such anti-inflammatory properties. Also without being bound by theory, it is believed herein that inflammation may play a significant role in causing or worsening the liver damage observed in FLDs. It is understood herein that selective anti-inflammatory effects may be particularly useful.

In each of the foregoing and each of the following embodiments and clauses, it is to be understood that the formulae include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulae are to be understood to be a description of such hydrates and/or solvates, including pharmaceutically acceptable solvates.

In each of the foregoing and each of the following embodiments and clauses, it is also to be understood that the formulae include and represent each possible isomer, such as stereoisomers and geometric isomers, both individually and in any and all possible mixtures. In each of the foregoing and each of the following embodiments, it is also to be understood that the formulae include and represent any and all crystalline forms, partially crystalline forms, and non crystalline and/or amorphous forms of the compounds.

Illustrative derivatives include, but are not limited to, both those compounds that may be synthetically prepared from the compounds described herein, as well as those compounds that may be prepared in a similar way as those described herein, but differing in the selection of starting materials. It is to be understood that such derivatives may include prodrugs of the compounds described herein, compounds described herein that include one or more protection or protecting groups, including compounds that are used in the preparation of other compounds described herein.

It is to be understood that each of the foregoing and following embodiments and clauses may be combined in chemically relevant ways to generate subsets of the embodiments described herein. Accordingly, it is to be further understood that all such subsets are also illustrative embodiments of the invention described herein.

In reciting the foregoing and following embodiments and clauses, it is to be understood that all possible combinations of features, and all possible subgenera and sub-combinations are described.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the terms “alkenyl” and “alkynyl” each include a chain of carbon atoms, which is optionally branched, and include at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like Illustratively, such particularly limited length alkyl groups, including C₁-C₈, C₁-C₆, and C₁-C₄, and C₂-C₈, C₂-C₆, and C₂-C₄, and the like may be referred to as lower alkyl. It is to be further understood that in certain embodiments alkenyl and/or alkynyl may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃-C₄, and the like Illustratively, such particularly limited length alkenyl and/or alkynyl groups, including C₂-C₈, C₂-C₆, and C₂-C₄, and C₃-C₈, C₃-C₆, and C₃-C₄, and the like may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. In embodiments of the invention described herein, it is to be understood, in each case, that the recitation of alkenyl refers to alkenyl as defined herein, and optionally lower alkenyl. In embodiments of the invention described herein, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like. Illustratively, such particularly limited length alkylene groups, including C₁-C₈, C₁-C₆, and C₁-C₄, and C₂-C₈, C₂-C₆, and C₂-C₄, and the like may be referred to as lower alkylene. It is to be further understood that in certain embodiments alkenylene and/or alkynylene may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃-C₄, and the like. Illustratively, such particularly limited length alkenylene and/or alkynylene groups, including C₂-C₈, C₂-C₆, and C₂-C₄, and C₃-C₈, C₃-C₆, and C₃-C₄, and the like may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the invention described herein, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamate, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

As used herein, the term “carbonyl and derivatives thereof” includes the group C(O), C(S), C(NH) and substituted amino derivatives thereof.

As used herein, the term “carboxylic acid and derivatives thereof” includes the group CO₂H and salts thereof, and esters and amides thereof, and CN.

As used herein, the term “sulfinic acid or a derivative thereof” includes SO₂H and salts thereof, and esters and amides thereof.

As used herein, the term “sulfonic acid or a derivative thereof” includes SO₃H and salts thereof, and esters and amides thereof.

As used herein, the term “sulfonyl” includes alkylsulfonyl, alkenylsulfonyl, alkynylsulfonyl, heteroalkylsulfonyl, heteroalkenylsulfonyl, heteroalkynylsulfonyl, cycloalkylsulfonyl, cycloalkenylsulfonyl, cycloheteroalkylsulfonyl, cycloheteroalkenylsulfonyl, arylsulfonyl, arylalkylsulfonyl, arylalkenylsulfonyl, arylalkynylsulfonyl, heteroarylsulfonyl, heteroarylalkylsulfonyl, heteroarylalkenylsulfonyl, heteroarylalkynylsulfonyl, acylsulfonyl, and the like, each of which is optionally substituted.

As used herein, the term “phosphinic acid or a derivative thereof” includes P(R)O₂H and salts thereof, and esters and amides thereof, where R is alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted.

As used herein, the term “phosphonic acid or a derivative thereof” includes PO₃H₂ and salts thereof, and esters and amides thereof.

As used herein, the term “hydroxylamino and derivatives thereof” includes NHOH, and alkyloxylNH alkenyloxylNH alkynyloxylNH heteroalkyloxylNH heteroalkenyloxylNH heteroalkynyloxylNH cycloalkyloxylNH cycloalkenyloxylNH cycloheteroalkyloxylNH cycloheteroalkenyloxylNH aryloxylNH arylalkyloxylNH arylalkenyloxylNH arylalkynyloxylNH heteroaryloxylNH heteroarylalkyloxylNH heteroarylalkenyloxylNH heteroarylalkynyloxylNH acyloxy, and the like, each of which is optionally substituted.

As used herein, the term “hydrazino and derivatives thereof” includes alkylNHNH, alkenylNHNH, alkynylNHNH, heteroalkylNHNH, heteroalkenylNHNH, heteroalkynylNHNH, cycloalkylNHNH, cycloalkenylNHNH, cycloheteroalkylNHNH, cycloheteroalkenylNHNH, arylNHNH, arylalkylNHNH, arylalkenylNHNH, arylalkynylNHNH, heteroarylNHNH, heteroarylalkylNHNH, heteroarylalkenylNHNH, heteroarylalkynylNHNH, acylNHNH, and the like, each of which is optionally substituted.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups, also referred to herein as aryl substituents or heteroaryl substituents, respectively, illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{x}, where x is an integer from 0-6 and Z^{x} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{x} is selected from —CO₂R⁴ and —CONR₅R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. Additional details of prodrug use are described in U.S. Pat. No. 5,627,165. It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as —OH—, —SH, —CO₂H, —NR₂. Illustrative prodrugs include but are not limited to carboxylate esters where the group is alkyl, aryl, arylalkyl, heteroaryl, heteroarylalkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. Illustrative esters, also referred to as active esters, include but are not limited to 1-indanyl, N-oxysuccinimide; acyloxyalkyl groups such as acetoxymethyl, pivaloyloxymethyl, β-acetoxyethyl, β-pivaloyloxyethyl, 1-(cyclohexylcarbonyloxy)prop-1-yl, (1-aminoethyl)carbonyloxymethyl, and the like; alkoxycarbonyloxyalkyl groups, such as ethoxycarbonyloxymethyl, α-ethoxycarbonyloxyethyl, β-ethoxycarbonyloxyethyl, and the like; dialkylaminoalkyl groups, including di-lower alkylamino alkyl groups, such as dimethylaminomethyl, dimethylaminoethyl, diethylaminomethyl, diethylaminoethyl, and the like; 2-(alkoxycarbonyl)-2-alkenyl groups such as 2-(isobutoxycarbonyl) pent-2-enyl, 2-(ethoxycarbonyl)but-2-enyl, and the like; and lactone groups such as phthalidyl, dimethoxyphthalidyl, and the like.

Further illustrative prodrugs contain a chemical moiety, such as an amide or phosphorus group functioning to increase solubility and/or stability of the compounds described herein. Further illustrative prodrugs for amino groups include, but are not limited to, (C₃-C₂₀)alkanoyl; halo-(C₃-C₂₀)alkanoyl; (C₃-C₂₀)alkenoyl; (C₄-C₇)cycloalkanoyl; (C₃-C₆)-cycloalkyl(C₂-C₁₆)alkanoyl; optionally substituted aroyl, such as unsubstituted aroyl or aroyl substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; optionally substituted aryl(C₂-C₁₆)alkanoyl and optionally substituted heteroaryl(C₂-C₁₆)alkanoyl, such as the aryl or heteroaryl radical being unsubstituted or substituted by 1 to 3 substituents selected from the group consisting of halogen, (C₁-C₃)alkyl and (C₁-C₃)alkoxy, each of which is optionally further substituted with 1 to 3 halogen atoms; and optionally substituted heteroarylalkanoyl having one to three heteroatoms selected from O, S and N in the heteroaryl moiety and 2 to 10 carbon atoms in the alkanoyl moiety, such as the heteroaryl radical being unsubstituted or substituted by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms. The groups illustrated are exemplary, not exhaustive, and may be prepared by conventional processes.

It is understood that the prodrugs themselves may not possess significant biological activity, but instead undergo one or more spontaneous chemical reaction(s), enzyme-catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serves to improve drug efficacy or safety through improved oral bioavailability, pharmacodynamic half-life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

As used herein, the term “leaving group” refers to a reactive functional group that generates an electrophilic site on the atom to which it is attached such that nucleophiles may be added to the electrophilic site on the atom. Illustrative leaving groups include, but are not limited to, halogens, optionally substituted phenols, acyloxy groups, sulfonoxy groups, and the like. It is to be understood that such leaving groups may be on alkyl, acyl, and the like. Such leaving groups may also be referred to herein as activating groups, such as when the leaving group is present on acyl. In addition, conventional peptide, amide, and ester coupling agents, such as but not limited to PyBop, BOP-Cl, BOP, pentafluorophenol, isobutylchloroformate, and the like, form various intermediates that include a leaving group, as defined herein, on a carbonyl group.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. In addition, it is to be understood that the compositions may be prepared from various co-crystals of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “administering” as used herein includes all means of introducing the compounds and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and/or vehicles.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustratively, administering includes both systemic and local use, such as when administered locally to the site of disease, injury, or defect, or to a particular organ or tissue system. Illustrative local administration may be performed during open surgery, or other procedures when the site of disease, injury, or defect is accessible. Alternatively, local administration may be performed using parenteral delivery where the compound or compositions described herein are deposited locally to the site without general distribution to multiple other non-target sites in the host animal being treated. It is further appreciated that local administration may be directly in the injury site, or locally in the surrounding tissue. Similar variations regarding local delivery to particular tissue types, such as organs, and the like, are also described herein.

Depending upon the disease as described herein, the route of administration and/or whether the compounds and/or compositions are administered locally or systemically, a wide range of permissible dosages are contemplated herein, including doses falling in the range from about 1 μg/kg to about 1 g/kg. The dosages may be single or divided, and may administered according to a wide variety of protocols, including q.d., b.i.d., t.i.d., or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

The effective use of the compounds, compositions, and methods described herein for treating or ameliorating one or more effects of a FLD or pulmonary fibrosis using one or more compounds described herein may be based upon animal models, such as murine, canine, porcine, and non-human primate animal models of disease. For example, it is understood that FLD or pulmonary fibrosis in humans may be characterized by a loss of function, and/or the development of symptoms, each of which may be elicited in animals, such as mice, and other surrogate test animals, such as those described herein.

The following examples further illustrate specific embodiments of the invention; however, the following illustrative examples should not be interpreted in any way to limit the invention.

### EXAMPLES

### Example

The following abbreviations are used herein: Alanine aminotransferase (ALT); Hematoxylin and eosin (HE); High fat diet (HFD); Murine leukemia virus reverse transcriptase (MMLV-RT); Nonalcoholic fatty liver disease (NAFLD); NAFLD Activity score (NAS); Non-alcoholic steatohepatitis (NASH); Standard deviation (SD); Specific pathogen-free (SPF); Stelic Animal Model (STAM); and Streptozotocin (STZ).

### Example

Illustratively, test compounds are formulated in vehicle, such as in 0.5% methycellulose+0.2% Tween® 80, and administered orally, such as in a volume of 10 mL/kg for each dose.

### Example

NASH-HCC (STAM™) Model. C57BL/6 (15-day-pregnant female) mice are obtained from Charles River Laboratories Japan (Kanagawa, Japan). Male pups are selected for the study. NASH is induced in male mice by a single subcutaneous injection of 200 μg streptozotocin (STZ, Sigma-Aldrich, USA) solution 2 days after birth, followed by feeding a HFD (57 kcal % fat, cat#: HFD32, CLEA Japan, Japan) beginning at 4 weeks of age. At 5 weeks of age, mice are divided into a control group and one or more treated group (generally 8 mice per group). Animals are sacrificed at 9 weeks of age, and biochemical, histological, and gene expression analyses are performed.

All animals used in the study are housed and cared for in accordance with local regulations. Test animals are maintained in an SPF facility under controlled conditions of temperature (23±2° C.), humidity (45±10%), lighting (12-hour artificial light and dark cycles; light from 8:00 to 20:00) and air exchange. A high pressure (20±4 Pa) is maintained in the experimental room. Test animals are housed in polycarbonate cages KN-600 (Natsume Seisakusho, Japan) with a maximum of 4 mice per cage. Sterilized PULMASμ (Material Research Center, Japan) are used for bedding and replaced once a week. Sterilized solid HFD is provided ad libitum, being placed in the metal lid on top of the cage. Pure water is provided ad libitum from a water bottle equipped with a rubber stopper and a sipper tube. Water bottles are replaced once a week, cleaned and sterilized in autoclave and reused.

Generally, elevated NAFLD activity score (NAS)≧5, steatosis, and elevated ALT is evident by week 6, NASH is evident by week 7, perisinusoidal fibrosis is evident by week 9, nodules are evident by week 12, and HCC is evident by week 16. It is to be understood herein that perisinusoidal fibrosis in mice is the model for NASH in humans (Fujii et al., Med Mol Morphol 46: 141-152 (2013)).

During weeks 5-9 (starting at day 35±2), test compound is administered (generally orally) at various doses diluted to 10 mL/kg and according to various dosing protocols to treatment groups (8 mice each) while a second group (8 mice) is administered vehicle only. Animals are sacrificed at 9 weeks.

### Example

Measurement of whole blood and plasma biochemistry. Non-fasting blood glucose was measured in whole blood using LIFE CHECK (EIDIA, Japan). For plasma biochemistry, blood was collected in polypropylene tubes with anticoagulant (Novo-Heparin, Mochida Pharmaceutical, Japan) and centrifuged at 1,000×g for 15 minutes at 4° C. The supernatant was collected and stored at −80° C. until use. Plasma ALT levels were measured by FUJI DRI-CHEM 7000 (Fujifilm, Japan). Plasma insulin, MIF and IL-22 were quantified by Ultra Sensitive Mouse Insulin ELISA Kit (Morinaga Institute of Biological science, Japan), Mouse Macrophage Migration Inhibitory Factor (MIF) ELISA (Kamiya Biomedical, USA), Quantikine ELISA Mouse/Rat IL-22 Immunoassay kit (R&D Systems, USA), respectively.

### Example

Measurement of serum biochemistry. For serum biochemistry, blood is collected in polypropylene tubes without anticoagulant and incubated at room temperature for 30 minutes, at 4° C. for 1 hour, and then centrifuged at 1,000×g for 15 minutes at 4° C. The supernatant is collected and stored at −80° C. until use. Serum triglyceride, HDL-cholesterol, LDL-cholesterol, VLDL-cholesterol, and chylomicron (ULDL) are quantified by HPLC at Skylight Biotech Inc. (Japan).

### Example

Measurement of liver biochemistry—liver triglyceride content. Liver total lipid-extracts are obtained by Folch's method (Folch J. et al., J. Biol. Chem., 1957; 226:497). Liver samples are homogenized in chloroform-methanol (2:1, v/v) and incubated overnight at room temperature. After washing with chloroform-methanol-water (8:4:3, v/v/v), the extracts are evaporated to dryness, and dissolved in isopropanol. Liver triglyceride (TG) and cholesterol contents were measured by Triglyceride E-test and Cholesterol E-test (Wako Pure Chemical Industries, Japan), respectively. Extracts are diluted 2-fold in isopropanol when the triglyceride content exceeds the detection limit.

### Example

Histopathological analyses. For HE staining, sections are cut from paraffin blocks of left lateral liver tissue prefixed in Bouin's solution and stained with Lillie-Mayer's Hematoxylin (Muto Pure Chemicals, Japan) and eosin solution (Wako Pure Chemical Industries). NAFLD Activity score (NAS) is calculated according to the criteria of Kleiner (Kleiner D E. et al., Hepatology, 2005; 41:1313). A NAS≧5 with steatosis and hepatocyte ballooning is generally considered diagnostic of NASH.

| Histological | | |
|---|---|---|
| feature | Score | Category definition |
| Steatosis | 0 | <5% |
| | 1 | 5-33% |
| | 2 | 34-66% |
| | 3 | >66% |

Plus

| | | |
|---|---|---|
| Hepatocyte | 0 | None |
| ballooning | 1 | Few |
| | 2 | Many |

Plus

| | | |
|---|---|---|
| Inflammation | 0 | None |
| | 1 | 1-2 foci per × 20 field |
| | 2 | 2-4 foci per × 20 field |
| | 3 | >4 foci per × 20 field |

NAS total 0-8

| | | |
|---|---|---|
| Fibrosis | 0 | No fibrosis |
| | 1a | Zone 3 mild perisinusoidal |
| | | fibrosis |
| | 1b | Zone 3 moderate perisinusoidal |
| | | fibrosis |
| | 1c | Periportal/portal fibrosis only |
| | 2 | Zone 3 + periportal/portal fibrosis |
| | 3 | Bridging fibrosis |
| | 4 | Cirrhosis |

Fibrosis score 0-4

### Example

To visualize collagen deposition (a measure of fibrosis), Bouin's fixed left lateral liver sections are stained using picro-Sirius red solution (Waldeck, Germany).

### Example

For immunohistochemistry (a measure of inflammation), sections are cut from frozen liver tissues embedded in Tissue-Tek O.C.T. compound and fixed in acetone. Endogenous peroxidase activity is blocked using 0.03% H₂O₂ for 5 minutes, followed by incubation with Block Ace (Dainippon Sumitomo Pharma, Japan) for 10 minutes. The sections were incubated with a 200-fold dilution of anti-F4/80 antibody (BMA Biomedicals, Switzerland), a 50-fold dilution of anti-CK-18 antibody (LifeSpan BioSciences, USA) or anti-Gr-1 antibody (culture supernatant) over night at 4° C. F4/80 is a marker for macrophage and Kupffer cells in the liver; CK-18 antibody measures the cytoskeleton in damaged hepatocytes; and anti-Gr-1 antibody measures the neutrophil response. After incubation with secondary antibody (HRP-Goat anti-rat antibody, Invitrogen, USA), enzyme-substrate reactions are performed using 3, 3′-diaminobenzidine/H2O2 solution (Nichirei, Japan).

### Example

For quantitative analysis of fibrosis area, bright field images of Sirius red-stained sections are captured around the central vein using a digital camera (DFC280; Leica, Germany) at 200-fold magnification, and the positive areas in 5 fields/section are measured using ImageJ software (National Institute of Health, USA). For quantitative analysis of inflammation area, bright field images of F4/80-immunostained sections are captured around the central vein using a digital camera (DFC280; Leica, Germany) at 200-fold magnification, and the positive areas in 5 fields/section are measured using ImageJ software (National Institute of Health, USA).

### Example

Statistical tests. Statistical analyses are performed using Student's t-test on GraphPad Prism 4 (GraphPad Software, USA), or equivalent device. P values <0.05 are considered statistically significant. A trend or tendency is assumed when a one-tailed t-test returned P values <0.10. Results are expressed as mean±SD. Statistical significance of vehicle treated groups generally refers to a comparison with normal groups, as appropriate. Statistical significance of groups treated with the compounds described herein generally refers to a comparison with vehicle treated groups, as appropriate.

### Example

Illustrative dose response test groups. Group 1-Normal. Eight normal mice are fed with a normal diet ad libitum without any treatment until 9 weeks of age. Group 2-Vehicle. Eight NASH mice are orally administered vehicle (0.5% w/v methylcellulose+0.2% Tween®80) in a volume of 10 mL/kg twice daily from 5 to 9 week of age. Group 3-solithromycin 5 mg/kg. Eight NASH mice are orally administered vehicle supplemented with solithromycin at a dose of 5 mg/kg in the morning and the vehicle in the evening (5 mg/kg/day) from 5 to 9 weeks of age. Group 4-solithromycin 10 mg/kg. Eight NASH mice are orally administered vehicle supplemented with solithromycin at a dose of 10 mg/kg twice daily (20 mg/kg/day) from 5 to 9 weeks of age. Group 5-solithromycin 25 mg/kg. Eight NASH mice are orally administered vehicle supplemented with solithromycin at a dose of 25 mg/kg twice daily (50 mg/kg/day) from 5 to 9 weeks of age. Group 6-solithromycin 50 mg/kg. Eight NASH mice are orally administered vehicle supplemented with solithromycin at a dose of 50 mg/kg once daily (50 mg/kg/day) from 5 to 9 weeks of age.

| | No. | | | Dose | Volume | | Sacrifice |
|---|---|---|---|---|---|---|---|
| Group | mice | Mice | Test substance | (mg/kg) | (mL/kg) | Regimens | (wks) |
| 1 | 8 | Normal | — | — | — | 5 wks-9 wks | 9 |
| 2 | 8 | STAM | Vehicle | — | 10 | Oral, twice per daily, | 9 |
| | | | | | | 5 wks-9 wks | |
| 3 | 8 | STAM | solithromycin + | 5 | 10 | Oral, twice per daily, | 9 |
| | | | Vehicle | | | 5 wks-9 wks | |
| 4 | 8 | STAM | solithromycin | 10 | 10 | Oral, twice per daily, | 9 |
| | | | | | | 5 wks-9 wks | |
| 5 | 8 | STAM | solithromycin | 25 | 10 | Oral, twice per daily, | 9 |
| | | | | | | 5 wks-9 wks | |
| 6 | 8 | STAM | solithromycin | 50 | 10 | Oral, once per daily, | 9 |
| | | | | | | 5 wks-9 wks | |

### Example

Mean body weight. The vehicle group showed a significant decrease in mean body weight on the day of sacrifice compared to the normal group. Group 4 showed a significant decrease in mean body weight compared to the vehicle group. There were no significant differences in mean body weight on the day of sacrifice between the vehicle group and any of Groups 3, 5, or 6. Without being bound by theory, it is believed herein that the decrease in mean body weight in Group 4 is not related to the dose or to the test compound and instead reflects the low number of test animals in the treatment group.

### Example

Liver weight. The vehicle group showed a significant increase (p<0.001) in mean liver weight compared to the normal group. Mean liver weight trended down as a function of increasing dose, though there was no statistically significant difference in liver weight between the vehicle group and the solithromycin 5 mg/kg group. The solithromycin 10 mg/kg and 25 mg/kg groups showed significant reductions (p<0.01 and p<0.001, respectively) in mean liver weight compared to the vehicle group. It is observed that the liver weight for the solithromycin 25 mg/kg group is numerically similar to the normal group. Without being bound by theory, it is believed herein that the compounds described herein are curative for this symptom of disease.

### Example

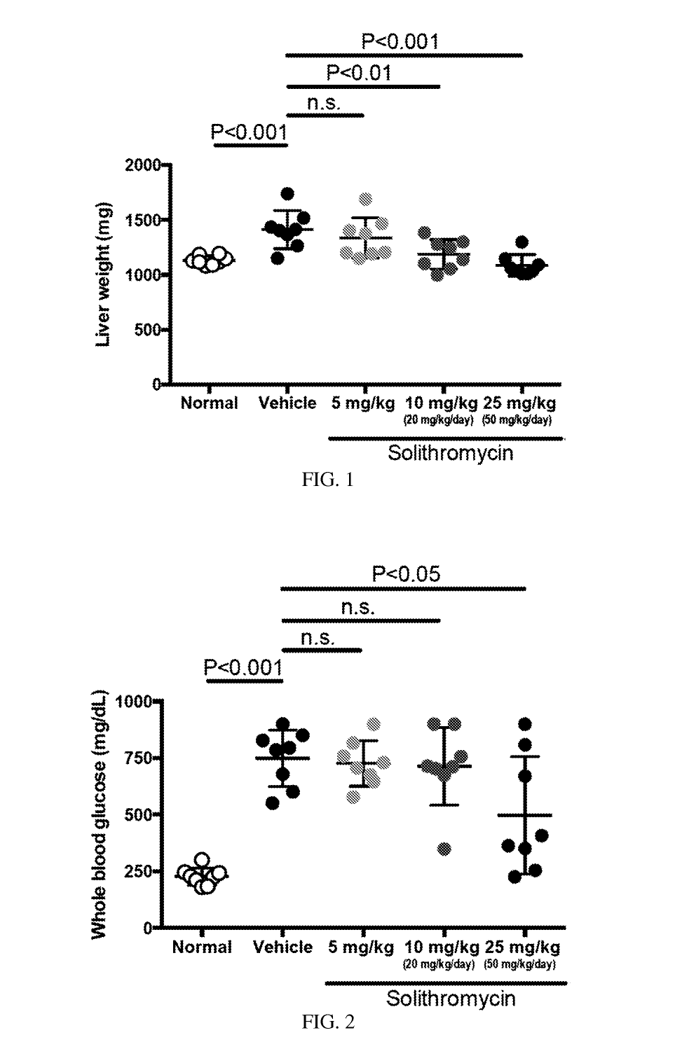

Liver-to-body weight ratio (FIG. 1). The vehicle group showed a significant increase (p<0.001) in mean liver-to-body weight ratio compared to the normal group. Mean liver-to-body weight ratio trended down as a function of increasing dose, though there were no statistically significant differences in ratio between the vehicle group and either the solithromycin 5 mg/kg or 10 mg/kg groups. The solithromycin 25 mg/kg group showed a significant decrease (p<0.01) in mean liver-to-body weight ratio compared to the vehicle group. Without being bound by theory, it is believed herein that decreasing mean liver weight is indicative of therapeutic efficacy.

| | | Liver-to-body | Whole Blood | |
|---|---|---|---|---|
| | Liver Weight | Weight Ratio | Glucose Levels | NAFLD |
| | (mg) | (%) | (mg/dL) | Activity Score |
| Vehicle | 1411 ± 173 | 7.3 ± 0.9 | 749 ± 125 | 5.4 ± 0.5 |
| Solithromycin | 1335 ± 185 | 7.0 ± 0.8 | 726 ± 101 | 4.8 ± 0.9 |
| (5 mg/kg q.d.) | | | | |
| Solithromycin | 1187 ± 134** | 6.7 ± 0.8 | 713 ± 172 | 3.3 ± 0.9** |
| (10 mg/kg b.i.d.) | | | | |
| Solithromycin | 1087 ± 97*** | 5.9 ± 0.7** | 497 ± 259* | 2.9 ± 1.0*** |
| (25 mg/kg b.i.d.) | | | | |
| Solithromycin | 1242 ± 141* | 6.0 ± 0.7* | 380 ± 170** | 3.0 ± 0.9**** |
| (50 mg/kg q.d.) | | | | |

Values are means ± SD,
*p < 0.05,
**p < 0.01,
***p < 0.001,
****p < 0.0001

### Example

Whole blood glucose (FIG. 2 and Table 1). The vehicle group showed a significant increase (p<0.001) in whole blood glucose levels compared to the normal group. Whole blood glucose levels trended down as a function of increasing dose, though there were no statistically significant differences in whole blood glucose levels between the vehicle group and either the solithromycin 5 mg/kg or 10 mg/kg groups. The solithromycin 25 mg/kg group showed a significant decrease (p<0.05) in whole blood glucose levels compared to the vehicle group. In addition, the decreases observed were not accompanied by an increase in insulin. Without being bound by theory, it is believed herein that decreasing whole blood glucose levels without increasing insulin is indicative of therapeutic efficacy for treating diabetes.

### Example

Plasma ALT (Table 1). Plasma ALT is reportedly an indicator of liver damage. Plasma ALT levels in the vehicle group tended to increase compared to the normal group. There were no significant differences in plasma ALT levels between the vehicle group and any doses of the solithromycin groups (Groups 3-6). Without being bound by theory, it is believed herein that the compounds described herein do not have a substantial effect on ALT, which would otherwise lead to unwanted side effects.

### Example

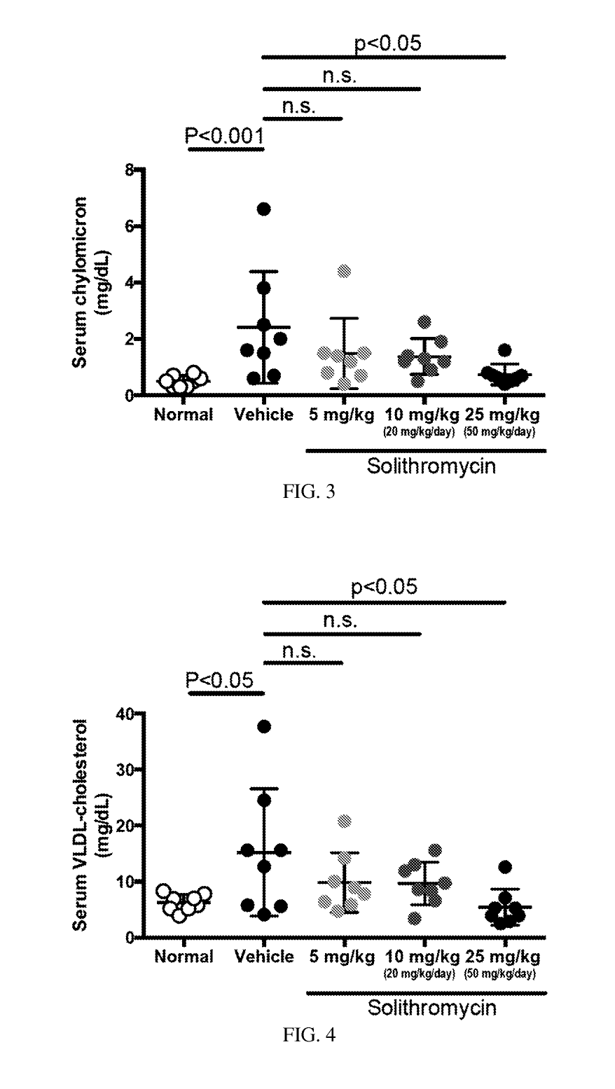

Serum chylomicron (FIG. 3 and Table 1). Serum chylomicron levels in the vehicle group were significantly increased (p<0.001) compared to the normal group. Serum chylomicron levels trended down as a function of increasing dose, though there were no statistically significant differences in serum chylomicron levels between the vehicle group and either the solithromycin 5 mg/kg or 10 mg/kg groups. Serum chylomicron levels in the solithromycin 25 mg/kg group were significantly decreased (p<0.05) compared to the vehicle group. Without being bound by theory, it is believed herein that decreasing serum chylomicron levels are indicative of therapeutic efficacy.

### Example

Serum VLDL-cholesterol (FIG. 4 and Table 1). Serum VLDL-cholesterol levels in the vehicle group was significantly increased (p<0.05) compared to the normal group. Serum VLDL-cholesterol levels trended down as a function of increasing dose, though there were no statistically significant differences in serum VLDL-cholesterol levels between the vehicle group and either the solithromycin 5 mg/kg or 10 mg/kg groups. Serum VLDL-cholesterol levels in the solithromycin 25 mg/kg group was significantly decreased (p<0.05) compared to the vehicle group. Without being bound by theory, it is believed herein that decreasing serum VLDL-cholesterol levels are indicative of therapeutic efficacy. It is observed that the serum VLDL-cholesterol level for the solithromycin 25 mg/kg group is numerically similar to the normal group. Without being bound by theory, it is believed herein that the compounds described herein are curative for this symptom of disease.

### Example

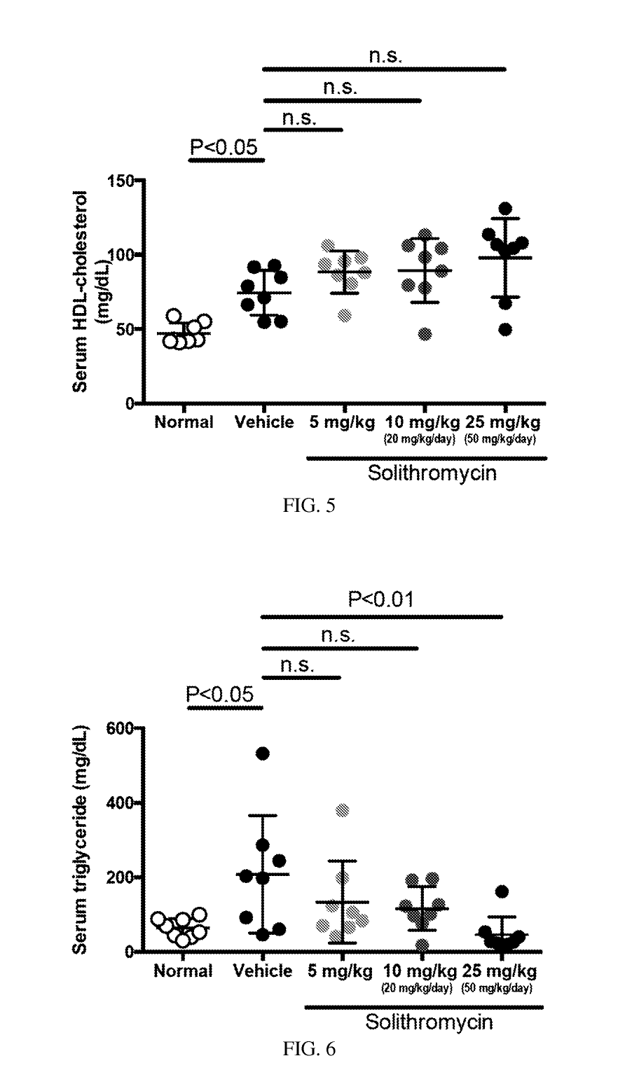

Serum HDL-cholesterol (FIG. 5 and Table 1). Serum HDL-cholesterol levels in the vehicle group were significantly increased (p<0.05) compared to the normal group. Serum HDL-cholesterol levels trended up as a function of increasing dose, though there were no statistically significant differences in serum HDL-cholesterol levels between the vehicle group and either the solithromycin 5 mg/kg, 10 mg/kg, or 25 mg/kg groups. Without being bound by theory, it is believed herein that increasing serum HDL-cholesterol levels are indicative of therapeutic efficacy.

### Example

Serum triglyceride (FIG. 6 and Table 1). Serum triglyceride levels in the vehicle group were significantly increased (p<0.05) compared to the normal group. Serum triglyceride levels trended down as a function of increasing dose, though there were no statistically significant differences in serum triglyceride levels between the vehicle group and either the solithromycin 5 mg/kg or 10 mg/kg groups. Serum triglyceride levels in the solithromycin 25 mg/kg group were significantly decreased (p<0.01) compared to the vehicle group. Without being bound by theory, it is believed herein that decreasing serum triglyceride levels are indicative of therapeutic efficacy. It is observed that the serum triglyceride level for the solithromycin 25 mg/kg group is numerically similar to the normal group. Without being bound by theory, it is believed herein that the compounds described herein are curative for this symptom of disease.

### Example

Liver triglyceride content (Table 1). Liver triglyceride content in the vehicle group was significantly increased (p<0.001) compared to the normal group. Liver triglyceride content trended down as a function of increasing dose, though there were no statistically significant differences in liver triglyceride content between the vehicle group and any doses (Groups 3-6) of the solithromycin groups. Without being bound by theory, it is believed herein that decreasing liver triglyceride levels are indicative of therapeutic efficacy.

### Example

Plasma insulin (Table 1). Plasma insulin levels in the vehicle group were significantly decreased (p<0.01) compared to the normal group. There were no statistically significant differences in plasma insulin levels between the vehicle group and any doses of the solithromycin groups. Without being bound by theory, it is believed herein that the compounds described herein effect a lowering of blood glucose levels by an insulin-independent process, such as by inhibiting gluconeogenesis.

### Example

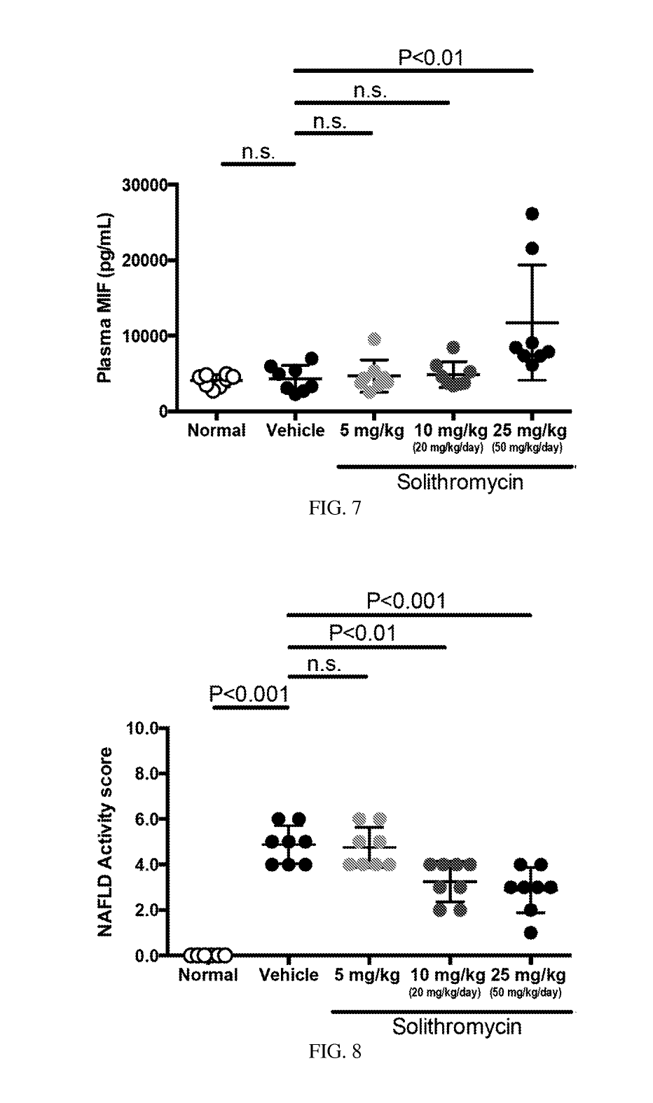

Plasma MIF (FIG. 7 and Table 1). There were no significant differences in plasma MIF levels between the vehicle group and the normal group. Plasma MIF levels trended up as a function of increasing dose, though there were no statistically significant differences in plasma MIF levels between the vehicle group and either the solithromycin 5 mg/kg or 10 mg/kg groups. Plasma MIF levels in the solithromycin 25 mg/kg group were significantly increased (p<0.01) compared to the vehicle group. MIF is reportedly an important regulator of innate immunity. White blood cells release MIF into the blood stream in an immune response. The circulating MIF binds to CD74 on other immune cells to trigger an acute immune response. Without being bound by theory, it is believed herein that MIF levels are a positive prognostic and diagnostic indicator of inflammation development and/or progression. In addition, though without being bound by theory, it is believed herein that MIF levels are a positive prognostic and diagnostic indicator of fibrosis, including liver fibrosis, development and/or progression. In addition, though without being bound by theory, it is believed herein that MIF levels are a positive prognostic and diagnostic indicator of HCC development and/or progression. In particular, reduced MIF levels are reportedly indicative of the potential for or presence or HCC. In addition, though without being bound by theory, it is believed herein that the data herein support the conclusion that the treatment methods described herein are efficacious in prophylactic treatment by delaying and/or preventing the progression to, or onset of fibrosis, including liver fibrosis, development and/or progression. In addition, though without being bound by theory, it is believed herein that the data herein support the conclusion that the treatment methods described herein are efficacious in prophylactic treatment by delaying and/or preventing the progression to, or onset of HCC.

### Example

Plasma IL-22 (Table 1). Plasma IL-22 levels in the vehicle group were significantly decreased (p<0.01) compared to the normal group. There were no statistically significant differences in plasma IL-22 levels between the vehicle group and any doses of the solithromycin groups.

TABLE 1
Biochemistry

| | | | SOLI | |
|---|---|---|---|---|
| | | SOLI | 10 mg/kg | SOLI |
| Parameter | Vehicle | 5 mg/kg q.d. | b.i.d. | 25 mg/kg b.i.d. |
| (mean ± SD) | (n = 8) | (n = 8) | (n = 8) | (n = 8) |
| Whole blood glucose | 749 ± 125 | 726 ± 101 | 713 ± 172 | 497 ± 259 |
| (mg/dL) | | | | |
| Plasma ALT (U/L) | 27 ± 20 | 52 ± 78 | 31 ± 19 | 34 ± 14 |
| Serum chylomicron | 2.4 ± 2.0 | 1.5 ± 1.2 | 1.4 ± 0.6 | 0.7 ± 0.4* |
| (mg/dL) | | | | |
| Serum HDL-cholesterol | 74.4 ± 15.1 | 88.4 ± 14.2 | 89.3 ± 21.5 | 97.9 ± 26.3 |
| (mg/dL) | | | | |
| Serum VLDL-cholesterol | 15.2 ± 11.4 | 9.8 ± 5.3 | 9.7 ± 3.8 | 5.4 ± 3.2* |
| (mg/dL) | | | | |
| Serum triglyceride | 208.2 ± 157.4 | 133.8 ± 110.1 | 116.5 ± 58.8 | 46.8 ± 47.8** |
| (mg/dL) | | | | |
| Liver triglyceride (mg/g | 132.0 ± 48.7 | 127.7 ± 62.3 | 161.8 ± 44.5 | 100.8 ± 35.3 |
| liver) | | | | |
| Plasma insulin (ng/mL) | 0.33 ± 0.29 | 0.25 ± 0.10 | 0.41 ± 0.35 | 0.41 ± 0.22 |
| Plasma MIF (pg/mL) | 4329 ± 1724 | 4665 ± 2131 | 4852 ± 1720 | 11748 ± 7633** |
| Plasma IL-22 (pg/mL) | 38.3 ± 10.5 | 31.8 ± 1.6 | 33.0 ± 5.0 | 33.8 ± 7.2 |

Values are means ± SD,
*p < 0.05,
**p < 0.01,
***p < 0.001,
****p < 0.0001

### Example

Histological analyses (FIG. 8). Compounds described herein, including solithromycin, significantly decrease the NAS of STAM mice, as measured by decreased steatosis, decreased hepatocyte ballooning, and decreased lobular inflammation. Because the NAS is one of the clinical endpoints for assessing the activity of NASH, the observed changes in the treatment group support the conclusion that compounds described herein are clinically efficacious as anti-NASH therapeutics (Sanyal A J. et al., Hepatology, 2011; 54:344). Photomicrographs of HE-stained sections are evaluated. Liver sections from the vehicle group exhibited severe microvesicular and macrovesicular fat deposition, hepatocellular ballooning and inflammatory cell infiltration. Consistent with these observations, the NAS significantly increased in the vehicle group compared to the normal group. All mice in Group 2 (vehicle only) had a NAS>5. There was no statistically significant difference in the NAS between the vehicle group and Group 3. Groups 4-6 showed significant improvements in hepatocellular ballooning and inflammatory cell infiltration, with significant reduction in the NAS compared to the vehicle group. The NAS for the solithromycin 10 mg/kg and 25 mg/kg groups were significantly decreased (p<0.01 and p<0.001, respectively) compared to the vehicle group.

### Example

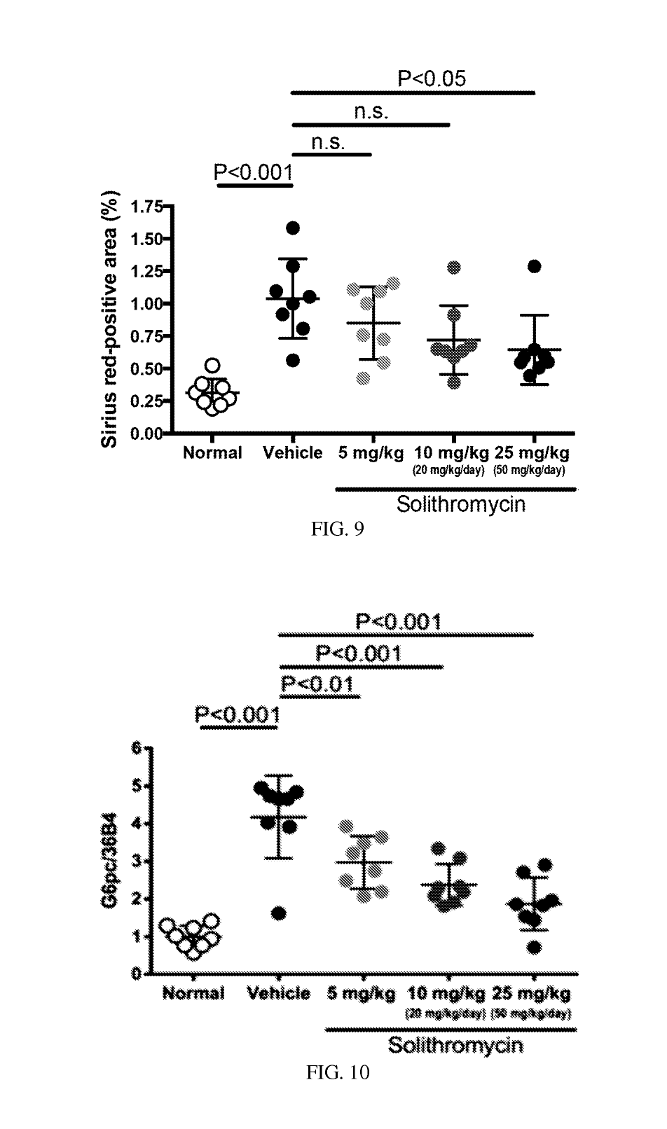

Sirius red staining (FIG. 9 and Table 2). Photomicrographs of Sirius red-stained sections of livers are evaluated. Liver sections from the vehicle group showed increased collagen deposition in the pericentral region of liver lobule compared to the normal group. The percentage of fibrosis area (Sirius red-positive area) significantly increased (p<0.001) in the vehicle group compared to the normal group. Compared to the vehicle group, the fibrosis area tended to decrease in Group 4, and significantly decreased in Groups 5 and 6. Fibrosis area trended down as a function of increasing dose, though there was no statistically significant differences in the fibrosis area between the vehicle group and the solithromycin 5 mg/kg and 10 mg/kg group. Fibrosis area in the solithromycin 25 mg/kg group was significantly decreased (p<0.05) compared to the vehicle group. Without being bound by theory, it is believed herein that that modest decrease in observed fibrosis in the treatment groups is due to low overall fibrosis in all groups. In longer term models, fibrosis will be more pronounced in the disease model. Without being bound by theory, it is believed herein that short-duration treatment shows a consistent improvement in fibrosis compared to untreated controls though fibrosis is not extensive in the model over short periods of time. However, long-term treatment shows continued improvement compared to untreated controls as the fibrosis increases over time. Without being bound by theory, it is believed herein that decreasing fibrosis area is indicative of therapeutic efficacy.

TABLE 2

| | | | SOLI | SOLI | SOLI |
|---|---|---|---|---|---|
| Parameter | Normal | Vehicle | 5 mg/kg | 10 mg/kg | 25 mg/kg |
| (mean ± SD) | (n = 8) | (n = 8) | (n = 8) | (n = 8) | (n = 8) |
| Sirius red-positive area | 0.31 ± 0.11 | 1.04 ± 0.31*** | 0.85 ± 0.28 | 0.72 ± 0.27 | 0.65 ± 0.27* |
| (% of area showing | | | | | |
| fibrosis) | | | | | |
| F4/80-positive area (%) | 5.33 ± 0.83 | 10.04 ± 1.86*** | 10.83 ± 1.38 | 9.33 ± 1.31 | 10.63 ± 2.11 |
| a measure of | | | | | |
| macrophage density | | | | | |

*p < 0.05,
**p < 0.01,
***p < 0.001

### Example

F4/80 Immunohistochemistry (Table 2). F4/80 antigen is a macrophage-restricted cell surface glycoprotein, where the stain is specific for macrophages. Photomicrographs of F4/80-immunostained sections are evaluated. Liver sections from the vehicle group showed an increased number and size of F4/80-positive cells in the liver lobule compared to the normal group. The percentage of F4/80-positive area significantly increased (p<0.001) in the vehicle group compared to the normal group. There were no significant differences in the area of F4/80-positive macrophages between the vehicle group and any of Groups 3-6. Without being bound by theory, it is believed herein that the compounds described herein do not have a substantial effect on macrophages, which would otherwise lead to unwanted side effects. It has been reported that solithromycin does not affect macrophage populations.

### Example

Gr-1 Immunohistochemistry as a neutrophil marker (FIG. 10). Photomicrographs of Gr-1-immunostained sections are evaluated. Liver sections from the vehicle group showed an increase in infiltrated Gr-1-positive cells in the liver lobule compared to the normal group. In all doses of the solithromycin treated groups, Gr-1 positive cells were decreased compared to the vehicle group. As described herein, the compounds do not appear to decrease neutrophil infiltration by either inhibiting macrophages, or mediating TNF alpha expression. Without being bound by theory, it is believed herein that decreasing neutrophil infiltration is indicative of therapeutic efficacy, including in both liver and lung diseases.

### Example

CK-18 Immunohistochemistry. Photomicrographs of CK-18-immunostained sections are evaluated. Liver sections from the vehicle group showed a strong intensity of immunostaining for CK-18 in the degenerative hepatocytes compare to the normal group. There were no obvious changes in CK-18 immunostaining between the vehicle group and any doses of the solithromycin groups. CK-18 is a major intermediate filament protein in the liver. Increased CK-18 staining reflects hepatocellular damage especially apoptosis. Without being bound by theory, it is believed herein that the compounds described herein do not show adverse effects on the cytoskeleton, as evidenced by CK-18 immunohistochemistry.

### Example

Quantitative RT-PCR. Total RNA is extracted from liver samples using RNAiso (Takara Bio, Japan) according to the manufacturer's instructions. One μg of RNA is reverse-transcribed using a reaction mixture containing 4.4 mM MgCl2 (Roche, Switzerland), 40 U RNase inhibitor (Toyobo, Japan), 0.5 mM dNTP (Promega, USA), 6.28 μM random hexamer (Promega), 5× first strand buffer (Promega), 10 mM dithiothreitol (Invitrogen) and 200 U MMLV-RT (Invitrogen) in a final volume of 20 μL. The reaction is carried out for 1 hour at 37° C., followed by 5 minutes at 99° C. Real-time PCR is performed using real-time PCR DICE and SYBR premix Taq (Takara Bio). To calculate the relative mRNA expression level, the expression of each gene is normalized to that of reference gene 36B4 (gene symbol: Rplp0). PCR-primer sets are described in copending U.S. provisional application No. 62/086,911, the disclosure of which is incorporated herein by reference.

### Example

Gene expression analysis (Table 3).

TNF-α mRNA expression levels were significantly up-regulated in the vehicle group compared to the normal group. There were no significant differences in TNF-α mRNA expression levels between the vehicle group and any doses of the solithromycin groups.

MCP-1 mRNA expression levels were significantly up-regulated (p<0.001) in the vehicle group compared to the normal group. MCP-1 mRNA expression levels trended down as a function of increasing dose, though there were no statistically significant differences in MCP-1 mRNA expression levels between the vehicle group and the solithromycin 5 mg/kg, 10 mg/kg, or 25 mg/kg groups. MCP-1 mRNA expression levels in the solithromycin 50 mg/kg q.d. group showed a significant decrease in MCP-1 mRNA expression levels in the liver compared with the vehicle group (normalized vehicle: 1.00±0.42, solithromycin: 0.64±0.14). As described herein, the compounds do not appear to decrease MCP-1 by either inhibiting macrophages, or mediating TNF alpha expression. Without being bound by theory, it is believed herein that decreasing MCP-1 mRNA expression levels are indicative of therapeutic efficacy, including in both liver and lung diseases.

MMP-9 mRNA expression levels were significantly up-regulated in the vehicle group compared to the normal group. MMP-9 mRNA expression levels trended down as a function of increasing dose, though there were no statistically significant differences in MMP-9 mRNA expression levels between the vehicle group and the solithromycin 5 mg/kg, 10 mg/kg, or 25 mg/kg groups. MMP-9 mRNA expression levels in the solithromycin 50 mg/kg q.d. group showed a significant decrease in MMP-9 mRNA expression levels in the liver compared with the vehicle group (normalized vehicle: 1.00±0.34, solithromycin: 0.59±0.25). As described herein, the compounds do not appear to decrease MMP-9 by either inhibiting macrophages, or mediating TNF alpha expression. Without being bound by theory, it is believed herein that decreasing MMP-9 mRNA expression levels are indicative of therapeutic efficacy, including in both liver and lung diseases.

Collagen Type 1 mRNA expression levels were significantly up-regulated in the vehicle group compared to the normal group. There were no significant differences in collagen Type 1 mRNA expression levels between the vehicle group and any doses of the solithromycin groups. Without being bound by theory, it is believed herein that the compounds described herein do not show adverse effects on the cytoskeleton, as evidenced by Collagen Type 1 mRNA expression levels.

Alpha-SMA mRNA expression levels were significantly up-regulated in the vehicle group compared to the normal group. There were no significant differences in α-SMA mRNA expression levels between the vehicle group and any doses of the solithromycin groups.

TIMP-1 mRNA expression levels tended to up-regulate in the vehicle group compared to the normal group. There were no significant differences in TIMP-1 mRNA expression levels between the vehicle group and any doses of the solithromycin groups.

TGF-β mRNA expression levels were significantly up-regulated in the vehicle group compared to the normal group. There were no significant differences in TGF-β mRNA expression levels between the vehicle group and any doses of the solithromycin groups.

Gck mRNA expression levels were significantly down-regulated in the vehicle group compared to the normal group. There were no significant differences in Gck mRNA expression levels between the vehicle group and any doses of the solithromycin groups.

G6pc mRNA expression levels were significantly up-regulated (p<0.001) in the vehicle group compared to the normal group (FIG. 10). G6pc mRNA expression levels were significantly down-regulated in all doses of the solithromycin groups compared to the vehicle group, as shown in Table 3. G6pc (glucose-6-phosphatase) is an integral membrane protein of the endoplasmic reticulum that catalyzes the hydrolysis of D-glucose 6-phosphate to D-glucose and orthophosphate. G6pc is a key enzyme in glucose homeostasis, in both gluconeogenesis and glycogenolysis. Without being bound by theory, it is believed herein that decreasing G6pc mRNA expression levels are indicative of therapeutic efficacy. Without being bound by theory, it is believed herein that the data supports the conclusion that the compounds described herein suppress and/or decrease gluconeogenesis, and are therefore, efficacious in treating diabetes. It is observed that the G6pc mRNA expression level for the solithromycin 25 mg/kg group is numerically similar to the normal group. Without being bound by theory, it is believed herein that the compounds described herein are curative for this symptom of disease.

Pck1 mRNA expression levels were significantly up-regulated in the vehicle group compared to the normal group. There were no significant differences in Pck1 mRNA expression levels between the vehicle group and any doses of the solithromycin groups.

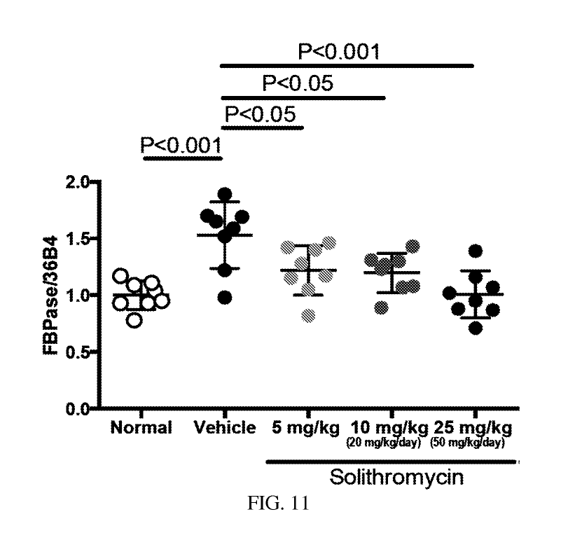

FBPase mRNA expression levels were significantly up-regulated (p<0.001) in the vehicle group compared to the normal group (FIG. 11). FBPase mRNA expression levels were significantly down-regulated in all doses of the solithromycin groups compared to the vehicle group, as shown in Table 3. FBPase (fructose bisphosphatase) converts fructose-1,6-bisphosphate to fructose 6-phosphate in gluconeogenesis, and catalyses the reverse of the reaction which is catalysed by phosphofructokinase in glycolysis. Without being bound by theory, it is believed herein that decreasing FBPase mRNA expression levels are indicative of therapeutic efficacy. Without being bound by theory, it is believed herein that the data supports the conclusion that the compounds described herein suppress and/or decrease gluconeogenesis, and are therefore, efficacious in treating diabetes. It is observed that the FBPase mRNA expression level for the solithromycin 25 mg/kg group is numerically similar to the normal group. Without being bound by theory, it is believed herein that the compounds described herein are curative for this symptom of disease.

Glut 2 mRNA expression levels were not significantly different between the vehicle group and the normal group. Glut 2 mRNA expression levels tended to down-regulated in the solithromycin 10 mg/kg group compared to the vehicle group. There were no significant differences in Glut 2 mRNA expression levels between the vehicle group and the other solithromycin groups.

TABLE 3
Gene expression analyses

| | | | SOLI | SOLI | SOLI |
|---|---|---|---|---|---|
| Parameter | Normal | Vehicle | 5 mg/kg | 10 mg/kg | 25 mg/kg |
| (mean ± SD) | (n = 8) | (n = 8) | (n = 8) | (n = 8) | (n = 8) |
| TNF-α/36B4 | 1.1 ± 0.6* | 2.8 ± 1.3 | 3.1 ± 1.2 | 3.0 ± 0.7 | 3.4 ± 1.3 |
| MCP-1/36B4 | 1.0 ± 0.4*** | 7.4 ± 3.4 | 7.2 ± 4.0 | 5.2 ± 1.9 | 4.9 ± 1.9 |
| MMP-9/36B4 | 1.0 ± 0.2** | 2.8 ± 1.5 | 2.9 ± 0.8 | 3.0 ± 1.1 | 2.6 ± 0.5 |
| Col1a2/36B4 | 1.0 ± 0.2** | 3.1 ± 1.2 | 3.6 ± 1.7 | 2.8 ± 1.0 | 2.9 ± 1.2 |
| α-SMA/36B4 | 1.1 ± 0.3* | 2.7 ± 1.2 | 3.8 ± 1.9 | 3.7 ± 1.0 | 3.3 ± 1.2 |
| Timp-1/36B4 | 1.0 ± 0.5 | 9.7 ± 4.0 | 16.0 ± 15.7 | 11.9 ± 6.8 | 11.0 ± 8.6 |
| TGF-β/36B4 | 1.0 ± 0.2*** | 2.1 ± 0.6 | 2.1 ± 0.4 | 1.8 ± 0.4 | 1.8 ± 0.5 |
| Gck/36B4 | 1.0 ± 0.3** | 0.5 ± 0.2 | 0.5 ± 0.2 | 0.4 ± 0.2 | 0.6 ± 0.4 |
| G6pc/36B4 | 1.0 ± 0.3*** | 4.2 ± 1.1 | 3.0 ± 0.7** | 2.4 ± 0.5*** | 1.9 ± 0.7*** |
| Pck1/36B4 | 1.0 ± 0.4* | 1.6 ± 0.4 | 1.5 ± 0.1 | 1.5 ± 0.4 | 1.3 ± 0.6 |
| FBPase/36B4 | 1.0 ± 0.1*** | 1.5 ± 0.3 | 1.2 ± 0.2* | 1.2 ± 0.2* | 1.0 ± 0.2*** |
| Glut 2/36B4 | 1.0 ± 0.2 | 1.0 ± 0.2 | 0.8 ± 0.1 | 0.8 ± 0.1 | 0.9 ± 0.1 |

vs Vehicle group,
*P < 0.05,
**P < 0.01,
***P < 0.001
36B4: Ribosomal protein, large, P0
TNF-α: Tumor necrosis factor,
MCP-1: Monocyte chemotactic protein, Chemokine (C-C motif) ligand,
MMP-9: Matrix metallopeptidase 9 (Mmp9),
Alpha-SMA: Actin, alpha 2, smooth muscle, aorta (Acta2),
TIMP-1: Tissue inhibitor of metalloproteinase 1 (Timp1), transcript variant 1,
Collagen Type 1: Collagen, type I, alpha 2 (Col1a2),
TGF-β: Transforming growth factor, beta 1 (Tgfb1),
Gck: Glucokinase (Gck),
G6pc: Glucose-6-phosphatase, catalytic (G6pc),
Pck1: Phosphoenolpyruvate carboxykinase 1, cytosolic (Pck1),
FBPase: Fructose bisphosphatase 1 (Fbp1),
Glut 2: Glucose transporter, type 2 (Glut 2)
TNF: Tumor necrosis factor,
MCP: Chemokine (C-C motif) ligand,
MMP-9: Matrix metallopeptidase 9 36
B4; Ribosomal protein, large, P0

### Example

Macroscopic liver appearance is improved with solithromycin treatment. Livers were removed from test animals and both the parietal side and the visceral side were visually evaluated for coloration and gross macroscopic appearance. Compared the normal group, the vehicle treated group livers were markedly yellow in color. The yellow coloration was generally homogenous to all parts of the liver, and similar on both the parietal side and the visceral side. As a function of increasing dose, the livers of the solithromycin treated group were substantially less yellow in color, and the yellow coloring was nearly absent at the highest solithromycin doses. In the highest solithromycin dosed group, the parietal side was nearly the same color as the normal group. In all solithromycin treated groups, the parietal side was slightly less yellow in color than the visceral side.

Solithromycin treatment significantly decreased the whole blood glucose levels and NAS, confirming its anti-NASH effects. In NAS, dose response of solithromycin was demonstrated as 25 mg/kg (twice daily) of solithromycin showed superior effect (p<0.001) compared to 10 mg/kg (twice daily, p<0.01) and 5 mg/kg (n.s.) of solithromycin. However, plasma insulin is not significantly affected by solithromycin. Therefore, solithromycin corrects high glucose with an insulin independent mechanism. Without being bound by theory, it is believed herein that solithromycin modulates gluconeogenesis. Without being bound by theory, it is believed herein that the glucose lowering effect of solithromycin is attributable to the suppression of gluconeogenesis in the liver, and therefore, solithromycin is useful in treating diabetes. Without being bound by theory, it is also believed herein that solithromycin treatment has anti-NASH and anti-fibrosis effects via modulating the glucose- and lipid-metabolism in this model.

Lobular inflammation and hepatocyte ballooning scores are prominent underlying factors in the anti-NASH mechanism of solithromycin. Although the areas of F4/80 and CK-18 seemed to be similar between tested groups, infiltration of neutrophils are reduced by the treatment with solithromycin.

Statistically significant blood glucose decrease is observed with solithromycin (25 mg/kg, b.i.d. and 50 mg/kg of, q.d.). Statistically significant reduced serum triglyceride and VLDL-cholesterol levels are observed with solithromycin (25 mg/kg, b.i.d.).

Statistically significant reduced G6pc and FBPase mRNA expression levels in the liver are observed with solithromycin (25 mg/kg, b.i.d.). No significant differences compare to vehicle treated controls were observed in relative gene expression of TNF-α, MMP-9, Gck, Pck1, Glut 2 for any of the lower doses (Groups 3-5). No significant differences compare to vehicle treated controls were observed in relative gene expression of genes associated with fibrosis regulation (TGF-β, collagen Type 1, α-SMA, TIMP-1) for any of the lower doses (Groups 3-5).

Statistically significant reduced fibrosis is observed with solithromycin (25 mg/kg, b.i.d.), as evidenced by sirius red staining.

### Example

Safety of solithromycin in humans with hepatic insufficiency. Solithromycin was determined to be well tolerated in humans with mild, moderate, and even severe hepatic impairment when administered at doses as high as 800 mg. No significant differences in safety were observed when compared to healthy controls given the same doses. In contrast, other macrolides, such as erythromycin and clarithromycin, are reportedly toxic to the liver, and as such are not compatible with treating liver diseases, and or host animals with hepatic insufficiency.

### Example

Safety and Pharmacokinetics of solithromycin in Subjects with Hepatic Impairment. The safety, pharmacokinetics (PK), and protein binding of solithromycin was evaluated in subjects with mild, moderate, and severe hepatic impairment compared to healthy subjects with normal hepatic function (matched for age, weight, and gender). The evaluation was performed in a Phase 1, open-label, multiple-dose study in subjects with mild (Child-Pugh Class A), moderate (Child-Pugh Class B), and severe (Child-Pugh Class C) hepatic impairment and healthy matched control subjects with normal hepatic function. All subjects received a once-daily dose of 800 mg on Day 1 followed by 400 mg on Days 2 through 5.

33 subjects were enrolled: 8 with mild impairment (mean Child-Pugh score 5.625), 8 with moderate impairment (mean Child-Pugh score 7.375), 8 with severe impairment (mean Child-Pugh score 10.625), and 9 healthy controls with normal hepatic function. One subject, a healthy control, discontinued study drug after 2 doses due to a rash; all other subjects (n=32) completed the study. Overall, the number of subjects reporting treatment-emergent AEs in the hepatic impaired cohorts (mild n=1, moderate n=4, severe n=4) was no greater than in the control group (n=4). The most commonly reported AEs were mild diarrhea and mild headache.

After 5 days of solithromycin administration, mean changes from baseline in liver function tests on Day 8 were not clinically significant in any cohort and did not differ significantly between cohorts. For ALT (IU/L), mean (±SD) changes by cohort: control=2.6±4.47, mild=4.0±8.00, moderate=7.8±6.92, severe=6.3±14.61. For AST (IU/L), mean (±SD) changes by cohort: control=−0.6±2.92, mild=0.4±5.93, moderate=0.1±10.56, severe=5.8±22.44. For direct bilirubin (mg/dL), mean (±SD) changes by cohort: control=0.00±0.053, mild=0.00±0.076, moderate=0.03±0.046, severe=0.04±0.207. No individual change from baseline in any liver function test was considered clinically significant.

PK parameters on Day 5 were compared between the hepatic impaired cohorts and the control group, and geometric mean ratios were calculated.

| | | | | Geometric Mean | 90% |
|---|---|---|---|---|---|
| | | Test | Reference | Ratio (%) | Confidence |
| Comparison | Parameter | Group | Group | (test/reference) | Intervals |
| Mild versus | Cmax (ng/mL) | 785.957 | 649.126 | 121.08 | 64.38-227.71 |
| Control | AUC0-t | 8872.658 | 7554.940 | 117.44 | 56.64-243.50 |
| | (ng * hr/mL) | | | | |
| | AUC0-tau | 7900.103 | 10491.88 | 75.30 | 47.89-118.39 |
| | (ng * hr/mL) | | | | |
| Moderate versus | Cmax (ng/mL) | 683.897 | 649.126 | 105.36 | 56.02-198.14 |
| Control | AUC0-t | 8902.279 | 7554.940 | 117.83 | 56.83-244.31 |
| | (ng * hr/mL) | | | | |
| | AUC0-tau | 7509.789 | 10491.88 | 71.58 | 45.52-112.55 |
| | (ng * hr/mL) | | | | |
| Severe versus | Cmax (ng/mL) | 504.311 | 649.126 | 77.69 | 41.31-146.11 |
| Control | AUC0-t | 8306.315 | 7554.940 | 109.95 | 53.03-227.95 |
| | (ng * hr/mL) | | | | |
| | AUC0-tau | 6175.788 | 10491.88 | 58.86 | 37.44-92.55 |
| | (ng * hr/mL) | | | | |

No accumulation was noted in any of the hepatic impaired cohorts on Day 5, though an increased half-life (h) was observed in the severe group (control=8.9, mild=10.2, moderate=10.4, severe=15.7). The mean plasma protein binding percentage, at Day 5 Cₘₐₓ, was not significantly affected by mild or moderate hepatic impairment, but was slightly lower in the severe cohort.

Macrolide antibiotics, like solithromycin, are primarily metabolized and excreted through liver-dependent mechanisms; this study evaluated the safety and PK of solithromycin in patients with chronic liver disease. It has been observed that approximately 78% of orally administered solithromycin is absorbed. Less than 15% unchanged solithromycin is excreted in the feces. It has also been observed that ˜70% of orally administered solithromycin is metabolized and excreted by the liver. Accordingly, high liver concentrations are observed. No dosage adjustment is needed when administering solithromycin to patients with mild, moderate, or severe hepatic impairment. Solithromycin was well tolerated in this patient population and no significant differences in safety, compared to healthy controls, were noted.

### Example

Bleomycin-Induced Lung Injury. Lung inflammation is induced in female mice by a single intratracheal administration of bleomycin. Twenty mice are divided into two groups. From Day −2 to Day 6, one group is administered vehicle and the other is administered test drug, such as solithromycin orally at a dose of 100 mg/kg. It is to be understood herein that 100 mg/kg in mice is generally considered to be equivalent to a 450-500 mg, such as 480 mg, dose in humans. Animals are sacrificed on Day 7.

Pathogen-free 7 weeks old female C57BL/6J mice are obtained from CLEA Japan (Tokyo, Japan) and allowed to acclimate at least 6 days. On day 0, twenty mice are induced to develop pulmonary fibrosis by a single intratracheal administration of bleomycin sulphate (BLM, Nippon Kayaku, Japan) in 0.9% saline in a volume of 50 μL per animal using a Microsprayer® (Penn-Century, USA). Individual body weight is measured daily during the experiment period. Survival, clinical signs and behavior of mice is monitored daily.

This same model may be performed with a prophylactic protocol, such as pretreatment for a period of time, for example 2 days, prior to inflammation induction.

### Example

Solithromycin Reduces Inflammation In Mice Caused By Bleomycin-Induced Lung Injury. Pulmonary fibrosis is a problem in the management of patients who have received chemotherapy for malignancies particularly with regimens that contain bleomycin (BLM), methotrexate, cyclophosphamide, and many new agents. These patients are susceptible to pulmonary infections as well as to inflammation from pulmonary injury. It has been discovered that solithromycin shows strong effects on cytokine release and superior anti-inflammatory effects. The effects of solithromycin on the ability to prevent lung inflammation and fibrosis is described in a bleomycin induced-lung inflammation and fibrosis model. Group 1 (BLM-Vehicle): Ten BLM-induced pulmonary fibrosis model mice are orally administered vehicle (Carboxymethyl cellulose) at a volume of 10 mL/kg once daily from day −2 (two days before BLM administration) to day 6. Group 2 (BLM-solithromycin): Ten BLM-induced pulmonary fibrosis model mice are orally administered vehicle supplemented with solithromycin at a dose of 100 mg/kg (dissolved in 0.5% methycellulose+0.2% Tween 80 vehicle) once daily from day −2 to day 6.

BALF samples were collected by flushing the lung via the trachea with sterile PBS three times (0.8 mL each). The first lavage was kept separate from the other two. BALF was centrifuged at 1,000×g for 3 minutes at 4° C. and the supernatant was collected and stored at −80° C. until use. The cell pellet from the first fraction and the remaining fractions of lavage fluid were pooled. Total cell number of BALF was counted with a hemocytometer and the cell differentials were determined by cytospin preparation stained with Diff-Quick (Sysmex, Japan). A differential cell count was performed on up to about 200 cells.

MMP-9 in the BALF was quantified by the Mouse Total MMP-9 Quantikine ELISA Kit (R&D Systems, USA). The immunoassay had a detection limit of 0.078 ng/mL

### Example

Histopathological Analyses. Right lung tissues prefixed in 10% neutral buffered formalin were sectioned and used for hematoxylin and eosin (HE) staining and Masson's Trichrome staining. The degree of pulmonary fibrosis was evaluated using the Ashcroft score (4) for the quantitative histological analysis.

### Example

Quantitative RT-PCR. Total RNA was extracted from lung samples using RNAiso (Takara Bio, Japan). One μg of RNA was reverse-transcribed using a reaction mixture containing 4.4 mM MgCl₂ (Roche, Switzerland), 40 U RNase inhibitor (Toyobo, Japan), 0.5 mM dNTP (Promega, USA), 6.28 μM random hexamer (Promega), 5× first strand buffer (Promega), 10 mM dithiothreitol (Invitrogen) and 200 U MMLV-RT (Invitrogen) in a final volume of 20 μL. The reaction was carried out for 1 hour at 37° C., followed by 5 minutes at 99° C. Real-time PCR was performed using real-time PCR DICE and SYBR premix Taq (Takara Bio). To calculate the relative mRNA expression level, the expression of each gene was normalized to that of reference gene GAPDH.

There were no significant differences in the body weight changes at any day and on the day of sacrifice between the vehicle group and the solithromycin group.

Analyses of BALF (bronchoalveolar lavage fluid). The cells in BALF are counted with a hemocytometer, and cell differentials are determined in cytospin preparations stained with Diff-Quik (Sysmex, Japan) MMP-9 in the supernatants from BALF are quantified by an enzyme-linked immunosorbent assay (ELISA; Cat# MMPT90, R&D Systems, USA). Histopathological assays for lung sections are performed according to standard methods. Masson's Trichrome staining and estimation of Ashcroft Score includes HE staining. Gene expression assays using total RNA from the lung are obtained using real-time RT-PCR analyses performed for TNF-α, MCP-1 and MMP-9.

The total numbers of cells in the bronchoalveolar lavage fluid (BALF), especially those of lymphocytes, neutrophils and eosinophils, are significantly decreased in the solithromycin group compared to the vehicle group. BALF There was no significant difference in the number of macrophages between the vehicle group and the solithromycin group. MMP-9 levels show a decreasing trend (P<0.1) in the solithromycin group.

### Example

Histological analysis: Masson's Trichrome staining and Ashcroft score. In the vehicle group, Masson's Trichrome staining revealed focal fibrotic lesions in the interstitial space of the lung. There was no significant difference in the Ashcroft score between the vehicle group and the solithromycin group (vehicle: 1.6±0.2, solithromycin: 1.4±0.5).

### Example

HE-staining. In the vehicle group, HE staining revealed alveolar wall thickening, diffuse alveolar destruction with collapse and obliteration of alveolar spaces, and inflammatory cell infiltration in the alveolar and interstitial space of lung. There were no obvious differences in the alveolar wall thickening, diffuse alveolar destruction and inflammatory cell infiltration between the vehicle group and the solithromycin group.

### Example

BALF analysis of mice treated with vehicle or solithromycin 50 mg/kg q.d.

| BALF Analysis | Vehicle treated | SOLI treated |
|---|---|---|
| Lymphocytes | 7.9 ± 3.9 × 10⁴ | 3.9 ± 1.9 × 10⁴** |
| Macrophages | 22.6 ± 7.3 × 10⁴ | 16.9 ± 9.6 × 10⁴ |
| Neutrophil | 2.4 ± 1.3 × 10⁴ | 0.7 ± 0.4 × 10⁴*** |
| Eosinophil | 0.8 ± 0.5 × 10⁴ | 0.3 ± 0.2 × 10⁴** |
| Total cells | 3.4 ± 1.1 × 10⁵ | 2.2 ± 1.2 × 10⁵* |
| Relative MCP-1 mRNA | 1.00 ± 0.42 | 0.38 ± 0.32** |
| expression | | |
| MMP-9 expression | 0.62 ± 0.25 ng/mL | 0.37 ± 0.30 ng/mL** |
| Relative MMP-9 mRNA | 1.00 ± 0.48 | 0.42 ± 0.45** |
| expression | | |
| TNF-α mRNA expression | 1.00 ± 1.19 ng/mL | 4.39 ± 2.36 ng/mL*** |
| Relative TNF-α mRNA | 1.00 ± 0.39 | 2.45 ± 2.10* |
| expression | | |

Values are means ± SD.
*p < 0.05;
**p < 0.01,
***p < 0.001
*p = <0.05

Without being bound by theory, it is believed herein that the efficacy of the compounds described herein regarding fibrosis may be due at least in part to their ability to decrease one or more of neutrophil count, MMP-9 expression, and/or MCP-1 expression. MCP-1 and MMP-9 are involved in the recruitment of inflammatory cells. Without being bound by theory, it is also believed herein that the efficacy is not dependent upon macrophage inhibition or decreasing TNFα expression. Without being bound by theory, it is also believed herein that the efficacy of the compounds described herein regarding fibrosis may be due at least in part to their ability to increase TNFα expression. TNFα has been reported to repress disease development via inducing apoptosis of inflammatory cells (Rodvold, K. A., M. H. Gotfried, J. G. Still, K. Clark, and P. Fernandes. “Comparison of plasma, epithelial lining fluid, and alveolar macrophage concentrations of solithromycin in healthy adult subjects. Antimicrob Agts Chemother. 2012; 56:5076-5081.)

Treatment with compounds described herein results in the reduction of inflammatory cells in the BALF. One efficacious endpoint is lower white cell count. Solithromycin treatment up-regulated TNF-α expression in the lung, which may have induced apoptosis of inflammatory cells. The results suggest that compounds described herein will be beneficial to prevent disease progression and development of pulmonary fibrosis in host animals.

### Example

Sulfur mustard (SM) toxicity to cells in culture and inhalation toxicity in rats. Anesthetized rats are intratracheally exposed to SM by vapor inhalation. Rats are treated with test drug, such as at a dose of 10, 20, or 40 mg/kg, one hour prior to exposure, and every twenty-four hours thereafter. After one, three, or seven days of treatment with test drug, symptoms caused by SM are evaluated. One efficacious endpoint is protective effects on airway epithelial cells and macrophages from SM-induced cytotoxicity. The effects are validated by histopathology. Another efficacious endpoint is a dose dependent protection of the trachea in the treated group. Additional detailed of this animal model are generally described in Gao (2007 and Gao (2011).

### Example

FXR Signal Pathway. The therapeutic efficacy of the compounds described herein do not depend on the FXR Signal Pathway. Solithromycin is tested in reporter cell assays expressing a hybrid FXR receptor (Indigo Biosciences, PA). Agonist and antagonist activity is measured. Solithromycin is tested starting at 30 μM and continuing with 1:3 dilutions. Solithromycin (CEM-101) was surprisingly found to not show agonistic activity nor significant antagonistic activity in the human FXR assays. Solithromycin does not show evidence of cytotoxicity in the antagonist assays. Without being bound by theory, it is believed herein that dependence upon the FXR pathway for activity in treating the diseases described herein may lead to increased triglycerides (TGs) and low density lipids (LDL) as an unwanted side effects. For example, patients with various forms of FLD may be at risk for cardiovascular disease, which may be aggravated by increased triglycerides and low density lipids.

### Example

The compounds described herein, including solithromycin, are effective in treating host animals with high cardiovascular disease risk. It has been unexpected found that when dosed either orally or intravenously, solithromycin does not show any QT or tQT prolongation, or other negative QT effects. PK analysis showed that solithromycin achieved plasma levels as high as 2000-3000 ng/mL. In contrast, each of erythromycin, clarithromycin, azithromycin, and telithromycin are reported to be QT positive.

### Example

Susceptibility of Anaerobic Intestinal Bacteria. Compounds described herein do not affect Gram-negative enterics, such as Enterobacteriaceae, Gram-negative anaerobes, or endotoxin producing bacteria, and have a minimal effect on bowel flora.

| Organism (No.) | MIC90 (mcg/mL) |
|---|---|
| Bacteroides spp. including B. fragilis (22) | >64 |
| Prevotella spp. (10) | 4 |
| Porphyromonas spp. (10) | 0.06 |
| Peptostreptococcus spp. (10) | 0.25 |
| Clostridium spp. (10) | 0.06 |
| C. difficile (10) | >64 |

### Compound Examples

### CEM-101 (solithromycin, SOLI)

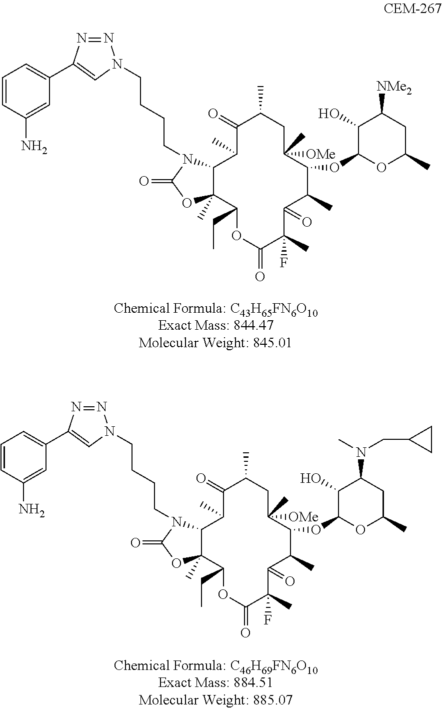 [SMILES: CC[C@H]1OC(=O)[C@@](C)(F)C(=O)[C@H](C)[C@@H](O[C@@H]2O[C@H](C)C[C@H](N(C)C)[C@H]2O)[C@@](C)(OC)C[C@@H](C)C(=O)[C@H](C)[C@H]2N(CCCCn3cc(-c4cccc(N)c4)nn3)C(=O)O[C@]12C ; CC[C@H]1OC(=O)[C@@](C)(F)C(=O)[C@H](C)[C@@H](O[C@@H]2O[C@H](C)C[C@H](N(C)CC3CC3)[C@H]2O)[C@@](C)(OC)C[C@@H](C)C(=O)[C@H](C)[C@H]2N(CCCCn3cc(-c4cccc(N)c4)nn3)C(=O)O[C@]12C]

The following publications, and each of the additional publications cited herein are incorporated herein by reference:

Gao X, Ray R, Xiao Y, Barker P E, Ray P. Inhibition of sulfur mustard-induced cytotoxicity and inflammation by the macrolide antibiotic roxithromycin in human respiratory epithelial cells. BMC Cell Biology 2007, http://www.biomedcentral.com/1471-2121/8/17

Gao X, Anderson D R, Brown A W, Lin H, Amnuaysirikul J, Chua A L, Holmes W W, and Ray P. Pathological studies on the protective effect of a macrolide antibiotic, roxithromycin, against sulfur mustard inhalation toxicity in a rat model pathological studies. Toxicol Pathol 2011; 39: 1056-1064. DOI: 10.1177/0192623311422079

1. A method for treating diabetes in a host animal, the method comprising the step of administering to the host animal an effective amount of a composition comprising one or more compounds of the formula 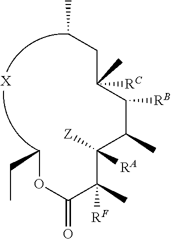
or a pharmaceutically acceptable salt thereof, or a hydroxyl or amino prodrug thereof; wherein:
X is a divalent radical selected from the group consisting of 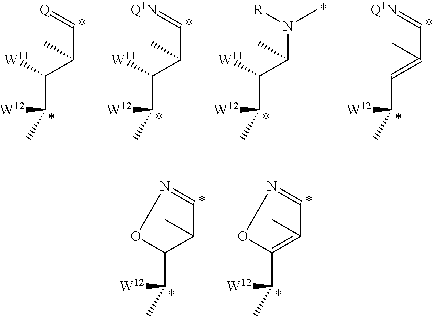
where X is connected at each (*) atom;
W¹¹ is hydroxy or a derivative thereof; W¹² is H, or hydroxy or a derivative thereof; or W¹¹ and W¹² are taken together with the attached carbon atoms to form an optionally substituted heterocycle containing oxygen or nitrogen or both oxygen and nitrogen;
Q is O or (NR, H); where R is hydrogen or optionally substituted alkyl; or R and W11 are taken together to form an aminal ether; and Q¹ is hydroxy or a derivative thereof or amino or a derivative thereof;
R^{A} is hydroxy or a hydroxy derivative, or a saccharide attached at oxygen; and Z is hydrogen; or R^{A} and Z are taken together with the attached carbon to form a C═O group;
R^{B} is an amino containing saccharide;
R^{C} is hydroxy or a derivative thereof; or
R^{C} and Q are taken together to form an enol ether; or
R^{C} and W¹² and Q are taken together to form a ketal; and
R^{F} is H or F.

2. A method for treating a FLD in a host animal, the method comprising the step of administering to the host animal an effective amount of a composition comprising one or more compounds of the formula 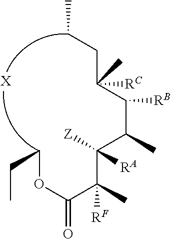
or a pharmaceutically acceptable salt thereof, or a hydroxyl or amino prodrug thereof; wherein:
X is a divalent radical selected from the group consisting of 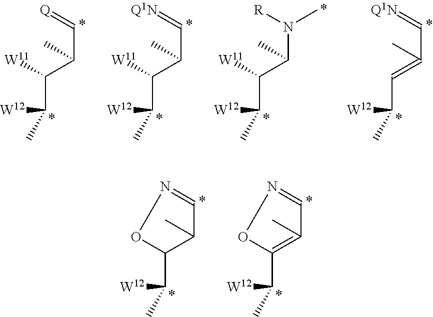
where X is connected at each (*) atom;
W¹¹ is hydroxy or a derivative thereof; W¹² is H, or hydroxy or a derivative thereof; or W¹¹ and W¹² are taken together with the attached carbon atoms to form an optionally substituted heterocycle containing oxygen or nitrogen or both oxygen and nitrogen;
Q is O or (NR, H); where R is hydrogen or optionally substituted alkyl; or R and W11 are taken together to form an aminal ether; and Q¹ is hydroxy or a derivative thereof or amino or a derivative thereof;
R^{A} is hydroxy or a hydroxy derivative, or a saccharide attached at oxygen; and Z is hydrogen; or R^{A} and Z are taken together with the attached carbon to form a C═O group;
R^{B} is an amino containing saccharide;
R^{C} is hydroxy or a derivative thereof; or
R^{C} and Q are taken together to form an enol ether; or
R^{C} and W¹² and Q are taken together to form a ketal; and
R^{F} is H or F.

3. The method of claim 2 wherein the FLD is NASH.

4. The method of claim 1 wherein at least one compound is of the formula 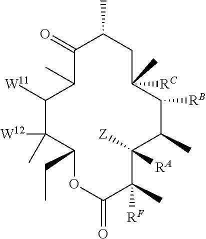
or a pharmaceutically acceptable salt thereof.

5. The method of claim 1 wherein at least one compound is of the formula 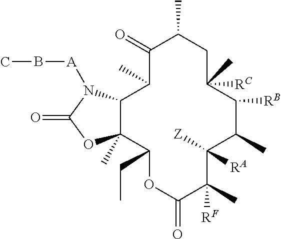
or a pharmaceutically acceptable salt thereof; where
C is H or alkyl, alkenyl, alkynyl, heteroalkyl, aryl, heteroaryl, arylalkyl, or heteroarylalkyl, each of which is optionally substituted;
B is a bond, or B is an optionally substituted heteroaryl; and
A is a bond, or A is an optional linker formed from one or more fragments selected from the group consisting of O, C(O), C, CR, CR₂, N, and NR, and combinations thereof, where each R is independently selected in each instance from the group consisting of hydrogen and optionally substituted alkyl.

6. The method of claim 1 wherein at least one compound is of the formula 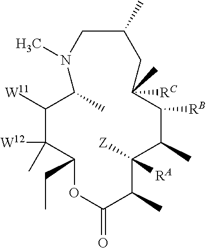
or a pharmaceutically acceptable salt thereof.

7. The method of claim 1 wherein Z is H and R^{A} is hydroxy or a derivative thereof.

8. The method of claim 1 wherein Z is H and R^{A} is cladinosyl.

9. The method of claim 1 wherein Z and R^{A} are taken together with the attached carbon to form C═O.

10. The method of claim 1 wherein R^{B} is desosaminyl.

11. The method of claim 1 wherein R^{B} is desmethyl desosaminyl, O-acyl desosaminyl, or O-alkyl desosaminyl.

12. The method of claim 1 wherein R^{B} is a radical of the formula 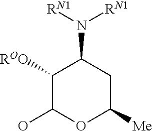
where each R^{N1} is independently selected in each instance from the group consisting of H and acyl, and alkyl, cycloalkyl, arylalkyl, and heteroarylalkyl, each of which is optionally substituted, providing that at least one R^{N1} is not methyl; or both R^{N1} are taken together with the attached nitrogen to form a nitrogen containing heterocycle; and R^{O} is H or acyl, or alkyl, cycloalkyl, arylalkyl, and heteroarylalkyl, each of which is optionally substituted; or R^{O} and one R^{N1} are taken together with the attached atoms to form an oxygen and nitrogen containing heterocycle.

13. The method of claim 1 wherein R^{B} is a radical of the formula

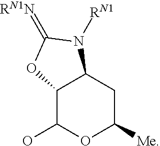

14. The method of claim 1 wherein R^{C} is hydroxy or alkoxy.

15. The method of claim 1 wherein R^{F} is F.

16. The method of claim 5 wherein A is alkylene.

17. The method of claim 5 wherein B is an imidazole radical or a 1,2,3-triazole radical.

18. The method of claim 5 wherein C is an optionally substituted heteroaryl or optionally substituted heteroarylalkyl radical.

19. The method of claim 1 wherein the compound is selected from the group consisting of solithromycin, roxithromycin, azithromycin, flurithromycin, and dirithromycin, and pharmaceutically acceptable salts thereof, and combinations thereof.

20. The method of claim 5 wherein C is optionally substituted aryl or optionally substituted arylalkyl.
